(19) United States
(12) Patent Application Publication
(10) Pub. No.: US 2020/0010457 A1
(43) Pub. Date: Jan. 9, 2020

(54) TETRAHYDROPYRAN AND TETRAHYDROTHIOPYRAN AMIDE DERIVATIVES HAVING MULTIMODAL ACTIVITY AGAINST PAIN

(71) Applicant: ESTEVE PHARMACEUTICALS, S.A., Barcelone (ES)

(72) Inventors: Monica GARCIA-LOPEZ, Barcelone (ES); Carmen ALMANSA-ROSALES, Barcelone (ES)

(21) Appl. No.: 16/469,733

(22) PCT Filed: Dec. 15, 2017
(86) PCT No.: PCT/EP2017/001430

§ 371 Date: Jun. 14, 2019

(30) Foreign Application Priority Data
Dec. 16, 2016 (EP) 16382606.8

(51) Int. Cl. C07D 405/12

(52) U.S. Cl. CPC C07D 405/12

(57) ABSTRACT

The present invention relates to tetrahydropyran and tetrahydrothiopyran amide derivatives having dual pharmacological activity towards both the sigma (σ) receptor, and the μ-opioid receptor, to processes of preparation of such compounds, to pharmaceutical compositions comprising them, and to their use in therapy, in particular for the treatment of pain.

### FIELD OF THE INVENTION

The present invention relates to tetrahydropyran and tetrahydrothiopyran amide derivatives of formula (I) having dual pharmacological activity towards both the sigma (σ) receptor, and the μ-opioid receptor, to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved [Turk D C, Wilson H D, Cahana A. Treatment of chronic non-cancer pain. Lancet 377, 2226-2235 (2011)]. Pain affects a big portion of the population with an estimated prevalence of around 20% and its incidence, particularly in the case of chronic pain, is increasing due to the population ageing. Additionally, pain is clearly related to comorbidities, such as depression, anxiety and insomnia, which lead to important productivity losses and socio-economic burden [Goldberg D S, McGee S J. Pain as a global public health priority. BMC Public Health. 11, 770 (2011)]. Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

As mentioned before, there are few available therapeutic classes for the treatment of pain, and opioids are among the most effective, especially when addressing severe pain states. They act through three different types of opioid receptors (mu, kappa and gamma) which are transmembrane G-protein coupled receptors (GPCRs). Still, the main analgesic action is attributed to the activation of the μ-opioid receptor (MOR). However, the general administration of MOR agonists is limited due to their important side effects, such as constipation, respiratory depression, tolerance, emesis and physical dependence [Meldrum, M. L. (Ed.). Opioids and Pain Relief: A Historical Perspective. Progress in Pain Research and Management, Vol 25. IASP Press, Seattle, 2003]. Additionally, MOR agonists are not optimal for the treatment of chronic pain as indicated by the diminished effectiveness of morphine against chronic pain conditions. This is especially proven for the chronic pain conditions of neuropathic or inflammatory origin, in comparison to its high potency against acute pain. The finding that chronic pain can lead to MOR down-regulation may offer a molecular basis for the relative lack of efficacy of morphine in long-term treatment settings [Dickenson, A. H., Suzuki, R. Opioids in neuropathic pain: Clues from animal studies. Eur J Pain 9, 113-6 (2005)]. Moreover, prolonged treatment with morphine may result in tolerance to its analgesic effects, most likely due to treatment-induced MOR down-regulation, internalization and other regulatory mechanisms. As a consequence, long-term treatment can result in substantial increases in dosing in order to maintain a clinically satisfactory pain relief, but the narrow therapeutic window of MOR agonists finally results in unacceptable side effects and poor patient compliance.

The sigma-1 (σ₁) receptor was discovered 35 years ago and initially assigned to a new subtype of the opioid family, but later on and based on the studies of the enantiomers of SKF-10,047, its independent nature was established. The first link of the σ₁ receptor to analgesia was established by Chien and Pasternak [Chien C C, Pasternak G W. Sigma antagonists potentiate opioid analgesia in rats. Neurosci. Lett. 190, 137-9 (1995)], who described it as an endogenous anti-opioid system, based on the finding that σ₁ receptor agonists counteracted opioid receptor mediated analgesia, while σ₁ receptor antagonists, such as haloperidol, potentiated it.

Many additional preclinical evidences have indicated a clear role of the σ₁ receptor in the treatment of pain [Zamanillo D, Romero L, Merlos M, Vela J M. Sigma 1 receptor: A new therapeutic target for pain. Eur. J. Pharmacol, 716, 78-93 (2013)]. The development of the σ₁ receptor knockout mice, which show no obvious phenotype and perceive normally sensory stimuli, was a key milestone in this endeavour. In physiological conditions the responses of the σ₁ receptor knockout mice to mechanical and thermal stimuli were found to be undistinguishable from WT ones but they were shown to possess a much higher resistance to develop pain behaviours than WT mice when hypersensitivity entered into play. Hence, in the σ₁ receptor knockout mice capsaicin did not induce mechanical hypersensitivity, both phases of formalin-induced pain were reduced, and cold and mechanical hypersensitivity were strongly attenuated after partial sciatic nerve ligation or after treatment with paclitaxel, which are models of neuropathic pain. Many of these actions were confirmed by the use of σ₁ receptor antagonists and led to the advancement of one compound, S1RA, into clinical trials for the treatment of different pain states. Compound S1RA exerted a substantial reduction of neuropathic pain and anhedonic state following nerve injury (i.e., neuropathic pain conditions) and, as demonstrated in an operant self-administration model, the nerve-injured mice, but not sham-operated mice, acquired the operant responding to obtain it (presumably to get pain relief), indicating that σ₁ receptor antagonism relieves neuropathic pain and also address some of the comorbidities (i.e., anhedonia, a core symptom in depression) related to pain states.

Pain is multimodal in nature, since in nearly all pain states several mediators, signalling pathways and molecular mechanisms are implicated. Consequently, monomodal therapies fail to provide complete pain relief. Currently, combining existing therapies is a common clinical practice and many efforts are directed to assess the best combination of available drugs in clinical studies [Mao J, Gold M S, Backonja M. Combination drug therapy for chronic pain: a call for more clinical studies. J. Pain 12, 157-166 (2011)]. Hence, there is an urgent need for innovative therapeutics to address this unmet medical need.

As mentioned previously, opioids are among the most potent analgesics but they are also responsible for various adverse effects which seriously limit their use.

Accordingly, there is still a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

Thus, the technical problem can therefore be formulated as finding compounds that have an alternative or improved pharmacological activity in the treatment of pain.

In view of the existing results of the currently available therapies and clinical practices, the present invention offers a solution by combining in a single compound binding to two different receptors relevant for the treatment of pain. This was mainly achieved by providing the compounds according to the invention that bind both to the μ-opioid receptor and to the σ₁ receptor.

### SUMMARY OF THE INVENTION

The main object of the invention is in one aspect directed to tetrahydropyran and tetrahydrothiopyran amide derivatives having a dual activity binding to the σ₁ receptor and the μ-opioid receptor for use in the treatment of pain.

As this invention is aimed at providing a compound or a chemically related series of compounds which act as dual ligands of the σ₁ receptor and the μ-opioid receptor it is a very preferred embodiment if the compound has a binding expressed as Kᵢ which is preferably <1000 nM for both receptors, more preferably <500 nM, even more preferably <100 nM.

More particularly the main aspect of the invention refers to a compound of general Formula (I),

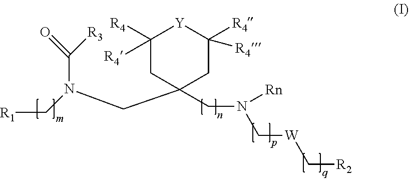

wherein R₁, R₂, R₃, R₄, R_{4′}, R_{4″}, R_{4″′}, Rₙ, Y, W, m, n, p and q are as defined below in the detailed description.

A further object of the invention refers to the processes for preparation of compounds of general formula (I).

A still further object of the invention refers to the use of some intermediate compounds for the preparation of a compound of general formula (I).

It is also an object of the invention a pharmaceutical composition comprising a compound of formula (I).

Finally, it is an object of the invention the use of compound as a medicament and more particularly for the treatment of pain and pain related conditions.

### DETAILED DESCRIPTION OF THE INVENTION

The invention is directed to a family of structurally distinct to tetrahydropyran and terahydrothiopyran amide derivatives which have a dual pharmacological activity towards both the sigma (σ) receptor and the μ-opioid receptor, thus solving the above problem of identifying alternative or improved pain treatments by offering such dual compounds.

The invention is directed to compounds having a dual activity binding to the σ₁ receptor and the μ-opioid receptor for use in the treatment of pain.

As this invention is aimed at providing a compound or a chemically related series of compounds which act as dual ligands of the σ₁ receptor and the μ-opioid receptor it is a preferred embodiment if the compound has a binding expressed as Kᵢ which is preferably <1000 nM for both receptors, more preferably <500 nM, even more preferably <100 nM.

The applicant has surprisingly found that the problem of providing a new effective and alternative for treating pain and pain related disorders can be solved by using a multimodal balanced analgesic approach combining two different synergistic activities in a single drug (i.e., dual ligands which are bifunctional and bind to μ-opioid receptor and to σ₁ receptor), thereby enhancing the opioid analgesia through the σ₁ activation without increasing the undesirable side effects. This supports the therapeutic value of a dual MOR/σ₁ receptor compound whereby the σ₁ receptor binding component acts as an intrinsic adjuvant of the MOR binding component.

This solution offered the advantage that the two mechanisms complement each other in order to treat pain and chronic pain using lower and better tolerated doses needed based on the potentiation of analgesia but avoiding the adverse events of μ-opioid receptor agonists.

A dual compound that possess binding to both the μ-opioid receptor and to the σ₁ receptor shows a highly valuable therapeutic potential by achieving an outstanding analgesia (enhanced in respect to the potency of the opioid component alone) with a reduced side-effect profile (safety margin increased compared to that of the opioid component alone) versus existing opioid therapies.

Advantageously, the dual compounds according to the present invention would in addition show one or more the following functionalities: σ₁ receptor antagonism and μ-opioid receptor agonism. It has to be noted, though, that both functionalities “antagonism” and “agonism” are also sub-divided in their effect into subfunctionalities like partial agonism or inverse agonism. Accordingly, the functionalities of the dual compound should be considered within a relatively broad bandwidth.

An antagonist on one of the named receptors blocks or dampens agonist-mediated responses. Known subfunctionalities are neutral antagonists or inverse agonists.

An agonist on one of the named receptors increases the activity of the receptor above its basal level. Known subfunctionalities are full agonists, or partial agonists.

In addition, the two mechanisms complement each other since MOR agonists are only marginally effective in the treatment of neuropathic pain, while σ₁ receptor antagonists show outstanding effects in preclinical neuropathic pain models. Thus, the σ₁ receptor component adds unique analgesic actions in opioid-resistant pain. Finally, the dual approach has clear advantages over MOR agonists in the treatment of chronic pain as lower and better tolerated doses would be needed based on the potentiation of analgesia but not of the adverse events of MOR agonists.

A further advantage of using designed multiple ligands is a lower risk of drug-drug interactions compared to cocktails or multi-component drugs, thus involving simpler pharmacokinetics and less variability among patients. Additionally, this approach may improve patient compliance and broaden the therapeutic application in relation to monomechanistic drugs, by addressing more complex aetiologies. It is also seen as a way of improving the R&D output obtained using the “one drug-one target” approach, which has been questioned over the last years [Bornot A, Bauer U, Brown A, Firth M, Hellawell C, Engkvist O. Systematic Exploration of Dual-Acting Modulators from a Combined Medicinal Chemistry and Biology Perspective. J. Med. Chem, 56, 1197-1210 (2013)].

In its broader aspect, the present invention is directed to compounds of general Formula (I):

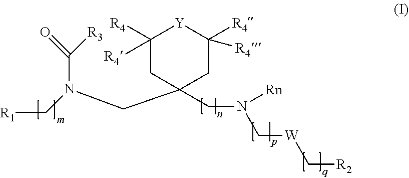

wherein

m is 0, 1, 2 or 3;

n is 0, 1, 2 or 3;

p is 1, 2 or 3;

q is 0, 1, 2 or 3;

Y is selected from —O— and —S—;

W is selected from —C(R_{w}R_{w′})—, —N(R_{w})—, and —O—;
wherein R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;
R_{w′} is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

R₃ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl, substituted or unsubstituted alkylcycloalkyl, substituted or unsubstituted alkylaryl and substituted or unsubstituted alkylheterocyclyl;

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R₄ and R_{4′}, may form together with the carbon atom to which they are attached a substituted or unsubstituted cycloalkyl;

R_{4″} and R_{4′″} are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R_{4″} and R_{4′″}, may form together with the carbon atom to which they are attached a substituted or unsubstituted cycloalkyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a further embodiment the compound according to the invention of general Formula (I) is a compound of general Formula (I′)

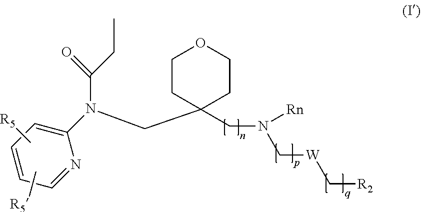

wherein R₂, R₅, R_{5′}, Rₙ, W, n, p and q are as defined in the description.

In a further embodiment the compound according to the invention of general Formula (I) is a compound of general Formula (I²′)

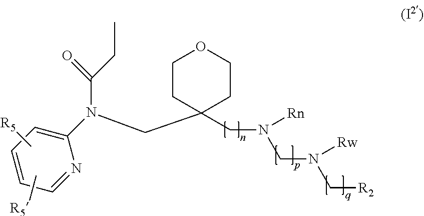

wherein R₂, R₅, R_{5′}, Rₙ, R_{w}, n, p and q are as defined in the description.

In a further embodiment the compound according to the invention of general Formula (I) is a compound of general Formula (I³′)

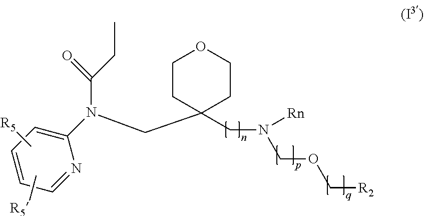

wherein R₂, R₅, R_{5′}, Rₙ, n, p and q are as defined in the description.

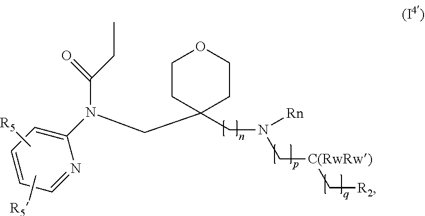

wherein R₂, R₅, R_{5′}, Rₙ, R_{w}, R_{w′}, n, p and q are as defined in the description.

For clarity purposes, all groups and definitions described in the description and referring to compounds of general Formula (I), also apply to compounds of general Formula (I′), (I²′), (l³′) or (I⁴′), as well as to all the intermediates of synthesis, when those groups are present in the mentioned general Markush formulae, since compounds of general Formulae (I′), (I²′), (l³′) or (I⁴′). are included in the general Formula (I).

For clarity purposes, the expression “the cycloalkyl in R₄—R_{4′}” means the cycloalkyl resulting when R₄ and R_{4′} form, together with the carbon to which they are attached, a cycloalkyl. This cycloalkyl can then be substituted or not. The same applies to R_{4″}—R_{4′″}.

This definition is also generally applicable and can be also applied as a definition of any other cycle (preferably cycloalkyl or heterocycl) formed from two different functional groups like e.g. “the cycle in Rᵢ—R_{i′}” means the cycle resulting when Rᵢ and R_{i′} form a cycle together with the atom(s) to which they are attached. This cycle can then be substituted or not.

For clarity purposes, the general Markush Formula (I)

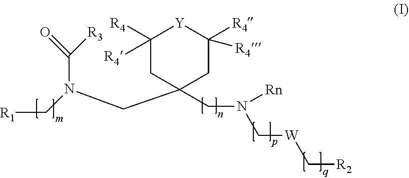

is equivalent to

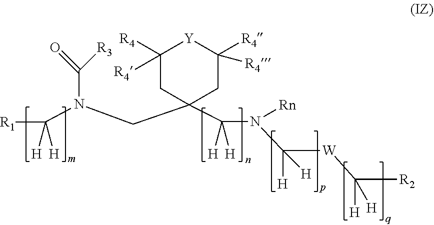

wherein only the —CH2- groups are included into the brackets and m, n, p or q mean the number of times that said —CH2- groups are repeated, respectively. The same would apply to general Markush Formulae (I), (I′), (I²′), (I³′) or (I⁴′) and to all intermediates of synthesis.

In addition, and for clarity purposes, it should further be understood that naturally if q is 0, R₂ and W are still present in general Markush Formula (I) or (I′), or if q is 0, R₂ and —N(R_{w})— are still present in general Markush Formula (I²′), or if q is 0, R₂ and —O— are still present in general Markush Formula (I³′), or or if q is 0, R₂ and —C(R_{w}R_{w′})— are still present in general Markush Formula (I⁴′).

It should also be understood that naturally if n is 0, —N(Rₙ)— is still present in general Markush Formulae (I), (I′), (I²′), (I³′) or (I⁴′).

It should also be understood that naturally if m is 0, R₁ and —NC(O)R₃ are still present in general Markush Formula (I).

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₅-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈alkyl like methyl, ethyl, propyl, butyl, pentyl, hexyl, heptyl, or octyl; preferably is C₁₋₆alkyl like methyl, ethyl, propyl, butyl, pentyl, or hexyl; more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆-alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—CH₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen (F, Cl, Br, I), —NR_{c}R_{c′}, —SR_{c}, —S(O)R_{c}, —S(O)R₂R_{c}, —OR_{c}, —C(O)O_{c}, —CN, —C(O)NR_{c}R_{c′}, haloalkyl, haloalkoxy or —OC₁₋₆ alkyl, being R_{c} represented by R₁₁, R₁₂, R₁₃, (being R_{c′} represented by R_{11′}, R_{12′}, R_{13′}, being R_{c″} represented by R_{11″}, R_{12″}, R₁₃″,) wherein R₁ to R_{14″} and R_{w}, R_{w′} and Rₙ are as defined in the description, and wherein when different radicals R₁ to R_{14″} and R_{w}, R_{w′} and Rₙ are present simultaneously in Formula I they may be identical or different.

Most preferably in connection with alkyl (also in alkylaryl, alkylheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which, if substituted, is substituted with one or more of halogen (F, Cl, Br, I), —OR_{c}, —CN, —SR_{c}, —S(O)R_{c}, —S(O)₂R_{c}, haloalkyl, haloalkoxy, —NR_{c}R_{c′}, or —OC₁₋₆alkyl, being R_{c} represented by R₁₁, R₁₂, R₁₃, (being R_{c′} represented by R_{11′}, R_{12′}, R_{13′}, being R_{c″} represented by R_{11″}, R_{12″}, R_{13″};), wherein R₁ to R_{14″} and R_{w}, R_{w″} and Rₙ are as defined in the description, and wherein when different radicals R₁ to R_{14″} and R_{w}, R_{w′} and Rₙ are present simultaneously in Formula I, they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OCCl₃, —OCF₃ and —OCH₂—CHCl₂. Preferably haloalkyl is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C_{3-4″} cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C_{4-6″} cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantly. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 5 to 18 membered mono or polycyclic ring systems with at least one aromatic ring but without heteroatoms even in only one of the rings. Examples are phenyl, naphthyl, fluoranthenyl, fluorenyl, tetralinyl, indanyl, 9H-fluorenyl or anthracenyl radicals, which can be unsubstituted or once or several times substituted. Most preferably aryl is understood in the context of this invention as phenyl, naphthyl or anthracenyl, preferably is phenyl.

A heterocyclyl radical or group (also called heterocyclyl hereinafter) is understood as meaning 5 to 18 membered mono or poly heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Examples include non-aromatic heterocyclyls such as tetrahydropyrane, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, benzothiazole, indole, benzotriazole, carbazole and quinazoline.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic 5 to 18 membered ring contains one or more heteroatoms selected from the group consisting of nitrogen, oxygen and/or sulfur in the ring; preferably is an aromatic 5 to 18 membered mono or polycyclic heterocyclic ring system of one or two rings of which at least one aromatic ring contains one or more heteroatoms selected from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring—with this (or these) ring(s) then not being aromatic—contains one or more heteroatoms selected from the group consisting of nitrogen, oxygen and/or sulfur in the ring; preferably is a 5 to 18 membered mono or polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, oxetane, especially is benzodioxane, morpholine, tetrahydropyran, piperidine, oxopyrrolidine, oxetane and pyrrolidine.

Preferably in the context of this invention heterocyclyl is defined as a 5 to 18 membered mono or polycyclic heterocyclic ring system of one or more saturated or unsaturated rings of which at least one ring contains one or more heteroatoms selected from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms selected from the group consisting of nitrogen, oxygen and/or sulfur in the ring.

Preferred examples of heterocyclyls include oxetane, oxazepane, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, thiophene, indole, benzimidazole, pyrrolo[2,3b]pyridine, benzoxazole, oxopyrrolidine, pyrimidine, oxazepane, oxetane and pyrrolidine.

In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) being connected to another atom through 1 to 4 (—CH₂—) groups. Most preferably alkylaryl is benzyl (i.e. —CH₂-phenyl).

In the context of this invention alkylheterocyclyl is understood as meaning an heterocyclyl group (see above) being connected to another atom through a C₁₋₆-alkyl (see above) which may be branched or linear and is unsubstituted or substituted once or several times. Preferably alkylheterocyclyl is understood as meaning a heterocyclyl group (see above) being connected to another atom through 1 to 4 (—CH₂—) groups. Most preferably alkylheterocyclyl is —CH₂-pyridine.

In the context of this invention alkylcycloalkyl is understood as meaning an cycloalkyl group (see above) being connected to another atom through a C₁₋₆-alkyl (see above) which may be branched or linear and is unsubstituted or substituted once or several times. Preferably alkylcycloalkyl is understood as meaning a cycloalkyl group (see above) being connected to another atom through 1 to 4 (—CH₂—) groups. Most preferably alkylcycloalkyl is —CH₂-cyclopropyl.

Preferably, the aryl is a monocyclic aryl. More preferably the aryl is a 5, 6 or 7 membered monocyclic aryl. Even more preferably the aryl is a 5 or 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —R_{c}, —OR_{c}, —CN, —NO₂, —C(O)OR_{c}, NR_{c}C(O)R_{c′}, —C(O)NR_{c}R_{c′}, —NR_{c}S(O)₂R_{c′}, ═O, —OCH₂CH₂OR_{c}, —NR_{c}C(O)NR_{c′}R_{c″}, —S(O)₂NR_{c}R_{c′}, —NR_{c}S(O)₂NR_{c′}R_{c″}, haloalkyl, haloalkoxy, —SR_{c}, —S(O)R_{c}, —S(O)₂R_{c} or —C(CH₃)OR_{c}; NR_{c}R_{c′}, with R_{c}, R_{c′} and R_{c″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C_{1-6—}alkyl (alkoxy); a saturated or unsaturated, linear or branched, substituted or unsubstituted —S—C₁₋₆-alkyl; a saturated or unsaturated, linear or branched, substituted or unsubstituted —C(O)—C₁₋₆₋alkyl-group; a saturated or unsaturated, linear or branched, substituted or unsubstituted —C(O)—O—C₁₋₆₋alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being R_{c} one of R₁₁, R₁₂ or R₁₄, (being R_{c′} one of R_{11′}, R_{12′} or R_{14′}; being one of R_{11″}, R_{12″} or R_{14″};), wherein R₁ to R_{14″} and R_{w}, R_{w′} and Rₙ are as defined in the description, and wherein when different radicals R₁ to R_{14″} and R_{w}, R_{w′} and Rₙ are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted is substituted (also in an alkylaryl, alkylcycloalkyl or alkylheterocyclyl) with one or more of halogen (F, Cl, Br, I), —R_{c}, —OR_{c}, —CN , —NO₂, —NR_{c}R_{c′″}, NR_{c}C(O)R_{c′}, —NR_{c}S(O)₂R_{c′}, ≡O, haloalkyl, haloalkoxy, or —C(CH₃)OR_{c}; —OC₁₋₄alkyl being unsubstituted or substituted with one or more of OR_{c} or halogen (F, Cl, I, Br), —CN, or —C₁₋₄alkyl being unsubstituted or substituted with one or more of OR_{c} or halogen (F, Cl, I, Br), being R_{c} one of R₁₁, R₁₂ or R₁₄, (being R_{c′} one of R_{11′}, R_{12′} or R_{14′}; being R_{c′} one of R_{11″}, R_{12″} or R_{14″};), wherein R₁ to R_{14″} and R_{w}, R_{w′} and Rₙ are as defined in the description, and wherein when different radicals R₁ to R_{14″} and R_{w}, R_{w′} and Rₙ are present simultaneously in Formula I they may be identical or different.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with

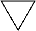

(leading to a spiro structure) and/or ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl is spirosubstituted or substituted with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with ≡O.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions.

The term “physiologically acceptable salt” means in the context of this invention any salt that is physiologically tolerated (most of the time meaning not being toxic—especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to general formula I defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to general formula I defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is an N-oxide of a compound according to the invention like a compound according to general formula I defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention.

The compounds of formula (I) as well as their salts or solvates of the compounds are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a more particular embodiment the compound according to the invention of general Formula (I)

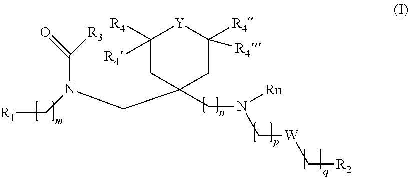

is a compound wherein

m is 0, 1, 2 or 3;

n is 0, 1, 2 or 3;

p is 1, 2 or 3;

q is 0, 1, 2 or 3;

Y is selected from —O— and —S—;

W is selected from —C(R_{w}R_{w′})—, —N(R_{w})—, and —O—;
wherein R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;
R_{w′} is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
wherein said cycloalkyl, aryl or heterocyclyl in R₁ if substituted, is substituted with one or more substituent/s selected from halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein the alkyl, alkenyl or alkynyl in R₁, if substituted, is substituted with one or more substituent/s selected from —OR₁₁, halogen, —CN, haloalkyl, haloalkoxy and —NR₁₁R_{11′};
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl,
wherein said cycloalkyl, aryl or heterocyclyl in R₂, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and C(CH₃)₂OR₁₂;
wherein the alkyl, alkenyl or alkynyl in R₂, if substituted, is substituted with one or more substituent/s selected from —OR₁₂, halogen, —CN, haloalkyl, haloalkoxy, —NR₁₂R_{12′};
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

R₃ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl, substituted or unsubstituted alkylcycloalkyl, substituted or unsubstituted alkylaryl and substituted or unsubstituted alkylheterocyclyl;

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R₄ and R_{4′}, may form together with the carbon atom to which they are attached a substituted or unsubstituted cycloalkyl;

R_{4″} and R_{4′″} are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R_{4″} and R_{4′″}, may form together with the carbon atom to which they are attached a substituted or unsubstituted cycloalkyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

the alkyl, alkenyl or alkynyl, other than those defined in R₁ or R₂, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;

the aryl, heterocyclyl or cycloalkyl other than those defined in R₁ or R₂, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and —C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1, 2 or 3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

n is 0, 1, 2 or 3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general f Formula (I) is a compound wherein

p is 1, 2 or 3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

q is 0, 1, 2 or 3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general f Formula (I) is a compound wherein

W is selected from —C(R_{w}R_{w′})—, —N(R_{w})— and —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general f Formula (I) is a compound wherein

W is —C(R_{w}R_{w′})—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general f Formula (I) is a compound wherein

W is —N(R_{w})—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general f Formula (I) is a compound wherein

W is —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

W is —C(R_{w}R_{w}^{′})—;
wherein R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;
R_{w}^{′} is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

W is —N(R_{w})—;
wherein R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted cycloalkyl, substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from hydrogen, substituted or unsubstituted C₁₋₆ alkyl, substituted or unsubstituted cycloalkyl, substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from hydrogen, substituted or unsubstituted C₁₋₆ alkyl, and substituted or unsubstituted aryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl, substituted or unsubstituted alkylcycloalkyl, substituted or unsubstituted alkylaryl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is selected from substituted or unsubstituted C₁₋₆ alkyl, substituted or unsubstituted cycloalkyl, substituted or unsubstituted aryl and substituted or unsubstituted heterocyclyl, substituted or unsubstituted alkylcycloalkyl, substituted or unsubstituted alkylaryl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄ and R_{4′} are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄ and R_{4′} are both hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄ and R_{4′}, may form together with the carbon atom to which they are attached a substituted or unsubstituted cycloalkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{4″} and R_{4′″} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{4″} and R_{4′″} are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{4′} and R_{4″′} are both hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{4″} and R_{4′″} may form together with the carbon atom to which they are attached a substituted or unsubstituted cycloalkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅ and R_{5′} are independently selected from hydrogen and haloalkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

Rₙ is selected from hydrogen, substituted or unsubstituted C₁₋₆ alkyl, substituted or unsubstituted C₂₋₆ alkenyl and substituted or unsubstituted C₂₋₆alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

Rₙ is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;
R_{w′} is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{w} is selected from hydrogen, substituted or unsubstituted C₁₋₆ alkyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{w} is selected from hydrogen, substituted or unsubstituted C₁₋₆ alkyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{w′} is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{w′} is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₁, R_{11′} and R_{11″} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₂, R_{12′} and R_{12″} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₂ is unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₃ and R_{13′} are independently selected from hydrogen, unsubstituted C₁₋₆ alkyl, unsubstituted C₂₋₆ alkenyl, and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₃ and R_{13′} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₄, R_{14′} and R_{14″} are independently selected from hydrogen, unsubstituted C₁₋₆ alkyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I), is a compound wherein

m is 0, 1, 2 or 3;
and/or

n is 0, 1, 2 or 3;
and/or

p is 1, 2 or 3;
and/or

q is 0, 1, 2 or 3;
and/or

Y is selected from —O— and —S—;
and/or

W is selected from —C(R_{w}R_{w′})—, —N(R_{w})— and —O—;
and/or

R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

wherein
the alkyl is C₁₋₆ alkyl like methyl, ethyl, propyl, butyl, pentyl, hexyl, isopropyl, or 2-methylpropyl; more preferably the alkyl is methyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or

R_{w′} is selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably the heterocyclyl is pyridine;
and/or

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl, ethyl or isopropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or

R₃ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl, substituted or unsubstituted alkylcycloalkyl, substituted or unsubstituted alkylaryl and substituted or unsubstituted alkylheterocyclyl;
wherein
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₅ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

and/or

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl,
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₄ and R_{4′} form together with the carbon atom to which they are attached a substituted or unsubstituted cycloalkyl;

wherein
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or

R_{4″} and R_{4′″} are independently selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R_{4′} and R_{4′″} may form together with the carbon atom to which they are attached a substituted or unsubstituted cycloalkyl;

wherein
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

and/or

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;

wherein
the alkyl is C₁₋₆ alkyl like methyl, ethyl, propyl, butyl, pentyl, hexyl, isopropyl, or 2-methylpropyl; more preferably the alkyl is methyl;

and/or

Rₙ is selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

and/or

R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

and/or

R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

wherein
the C₁₋₆ alkyl is preferably selected from methyl, ethyl, propyl, butyl, pentyl, hexyl, isopropyl, or 2-methylpropyl, preferably, C₁₋₆ alkyl is ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

and/or

R₁₃ and R_{13′} are independently selected from hydrogen, unsubstituted C₁₋₆ alkyl, unsubstituted C₂₋₆ alkenyl, and unsubstituted C₂₋₆ alkynyl;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

and/or

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{w} as defined in any of the embodiments of the present invention,
the alkyl is C₁₋₆ alkyl like methyl, ethyl, propyl, butyl, pentyl, hexyl, isopropyl, or 2-methylpropyl; more preferably the alkyl is methyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{w′} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably the heterocyclyl is pyridine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂ as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl, ethyl or isopropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃ as defined in any of the embodiments of the present invention,
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₅ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄ and R_{4′} as defined in any of the embodiments of the present invention,
the C₁₋₆ alkyl is preferably selected from methyl, ethyl, propyl, butyl, pentyl, hexyl, isopropyl, or 2-methylpropyl,
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄ and R_{4′} as defined in any of the embodiments of the present invention,
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{4″} and R_{4′″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{4″} and R_{4′″} as defined in any of the embodiments of the present invention,
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₅ and R_{5′} as defined in any of the embodiments of the present invention,
the alkyl is C₁₋₆ alkyl like methyl, ethyl, propyl, butyl, pentyl, hexyl, isopropyl, or 2-methylpropyl; more preferably the alkyl is methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in Rₙ as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₁, R_{11′} and R_{11″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₂, R_{12′} and R_{12″} as defined in any of the embodiments of the present invention,
the C₁₋₆ alkyl is preferably selected from methyl, ethyl, propyl, butyl, pentyl, hexyl, isopropyl, or 2-methylpropyl, preferably, the C₁₋₆ alkyl is ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₃ and R_{13′} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₄, R_{14′} and R_{14″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepane, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1, 2 or 3; preferably m is 0;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

n is 0, 1, 2 or 3; preferably n is 0, 2 or 3; more preferably n is 0 or n is 2 or 3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

p is 1, 2 or 3; preferably p is 1 or 2;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

q is 0, 1, 2 or 3; preferably q is 0 or 1;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

Y is —O— or —S—; preferably, Y is —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

W is —C(R_{w}R_{w′})—, —N(R_{w})— or —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I′)

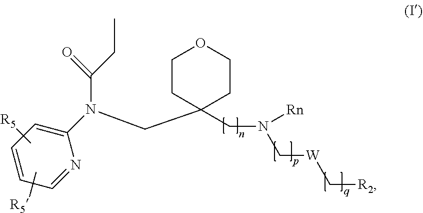

wherein

n is 0, 1, 2 or 3;

p is 1, 2 or 3;

q is 0, 1, 2 or 3;

W is selected from —C(R_{w}R_{w′})—, —N(R_{w})— and —O—;
wherein R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;
R_{w′} is selected from hydrogen, substituted or unsubstituted C₁₋₆ alkyl, substituted or unsubstituted C₂₋₆ alkenyl and substituted or unsubstituted C₂₋₆ alkynyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}—, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I′)

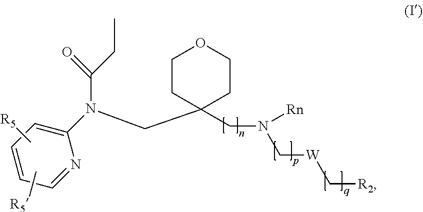

wherein

n is 0, 1, 2 or 3;

p is 1, 2 or 3;

q is 0, 1, 2 or 3;

W is selected from —C(R_{w}R_{w′})—, —N(R_{w})— and —O—;
wherein R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;
R_{w′} is selected from hydrogen, substituted or unsubstituted C₁₋₆ alkyl, substituted or unsubstituted C₂₋₆ alkenyl and substituted or unsubstituted C₂₋₆ alkynyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
wherein said cycloalkyl, aryl or heterocyclyl in R₂, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and C(CH₃)₂OR₁₂;
wherein the alkyl, alkenyl or alkynyl in R₂, if substituted, is substituted with one or more substituent/s selected from —OR₁₂, halogen, —CN, haloalkyl, haloalkoxy, —NR₁₂R_{12′};
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″′} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

the alkyl, alkenyl or alkynyl, other than those defined in R₂, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;

the aryl, heterocyclyl or cycloalkyl other than those defined in R₂, if substituted, is substituted with one or more substituents selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and —C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I²′)

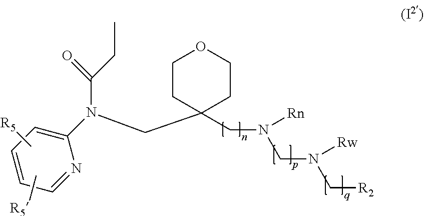

wherein

n is 0, 1, 2 or 3;

p is 1, 2 or 3;

q is 0, 1, 2 or 3;

R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I²′)

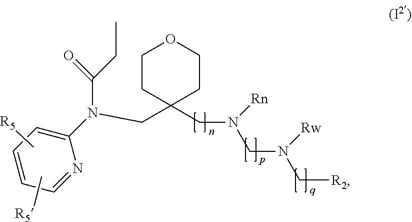

wherein

n is 0, 1, 2 or 3;

p is 1, 2 or 3;

q is 0, 1, 2 or 3;

R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
wherein said cycloalkyl, aryl or heterocyclyl in R₂, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and C(CH₃)₂OR₁₂;
wherein the alkyl, alkenyl or alkynyl in R₂, if substituted, is substituted with one or more substituent/s selected from —OR₁₂, halogen, —CN, haloalkyl, haloalkoxy, —NR₁₂R_{12′};
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

the alkyl, alkenyl or alkynyl, other than those defined in R₂, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;

the aryl, heterocyclyl or cycloalkyl other than those defined in R₂, if substituted, is substituted with one or more substituents selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and —C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I³′)

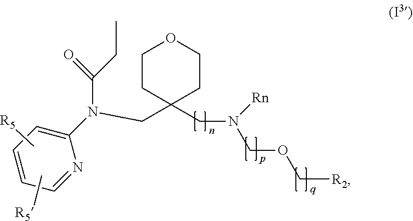

wherein

n is 0, 1, 2 or 3;

p is 1, 2 or 3;

q is 0, 1, 2 or 3;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I³′)

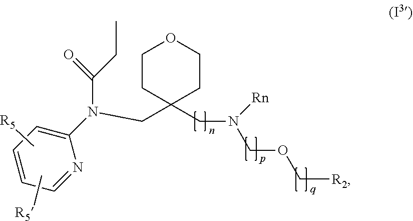

wherein

n is 0, 1, 2 or 3;

p is 1, 2 or 3;

q is 0, 1, 2 or 3;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
wherein said cycloalkyl, aryl or heterocyclyl in R₂, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and C(CH₃)₂OR₁₂;
wherein the alkyl, alkenyl or alkynyl in R₂, if substituted, is substituted with one or more substituent/s selected from —OR₁₂, halogen, —CN, haloalkyl, haloalkoxy, —NR₁₂R_{12′};
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

the alkyl, alkenyl or alkynyl, other than those defined in R₁ or R₂, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;

the aryl, heterocyclyl or cycloalkyl other than those defined in R₁ or R₂, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and —C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I⁴′)

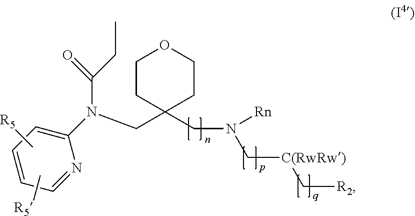

wherein

n is 0, 1, 2 or 3;

p is 1, 2 or 3;

q is 0, 1, 2 or 3;

R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

R_{w′} is selected from hydrogen, substituted or unsubstituted C₁₋₆ alkyl, substituted or unsubstituted C₂₋₆ alkenyl and substituted or unsubstituted C₂₋₆ alkynyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;

R₅ and R_{5′} are independently selected from hydrogen, halogen, —R₁₁, —OR₁₁, —NO₂, —NR₁₁R_{11″}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (l⁴′)

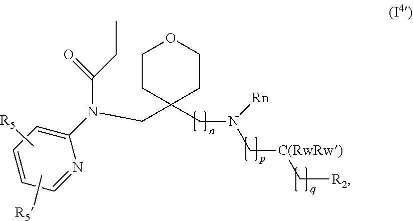

wherein

n is 0, 1, 2 or 3;

p is 1, 2 or 3;

q is 0, 1, 2 or 3;

R_{w} is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;

R_{w′} is selected from hydrogen, substituted or unsubstituted C₁₋₆ alkyl, substituted or unsubstituted C₂₋₆ alkenyl and substituted or unsubstituted C₂₋₆ alkynyl;

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
wherein said cycloalkyl, aryl or heterocyclyl in R₂, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR₁₂′R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and C(CH₃)₂OR₁₂;
wherein the alkyl, alkenyl or alkynyl in R₂, if substituted, is substituted with one or more substituent/s selected from —OR₁₂, halogen, —CN, haloalkyl, haloalkoxy, —NR₁₂R_{12′};
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

Rₙ is selected from hydrogen, substituted or unsubstituted C₁₋₆ alkyl, substituted or unsubstituted C₂₋₆ alkenyl and substituted or unsubstituted C₂₋₆ alkynyl;

the alkyl, alkenyl or alkynyl, other than those defined in R₁ or R₂, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;

the aryl, heterocyclyl or cycloalkyl other than those defined in R₁ or R₂, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR_{14′}R_{14″}, —OCH₂CH₂OR₁₄, —NR₁₄S(O)₂NR_{14′}R_{14″} and —C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

m is 0.

In a preferred embodiment

n is 0 or n is 2 or 3.

In a preferred embodiment

p is 1 or 2.

In a preferred embodiment

q is 0 or 1.

In a preferred embodiment

Y is —O—.

In a preferred embodiment

R_{w} is hydrogen, substituted or unsubstituted methyl or substituted or unsubstituted benzyl, preferably hydrogen, unsubstituted methyl or unsubstituted benzyl.

In a preferred embodiment

R_{w′} is hydrogen or substituted or unsubstituted methyl, preferably hydrogen unsubstituted methyl.

In a preferred embodiment

R_{w} is hydrogen, substituted or unsubstituted methyl or substituted or unsubstituted benzyl, preferably hydrogen, unsubstituted methyl or unsubstituted benzyl, while R_{w′} is hydrogen or substituted or unsubstituted methyl, preferably hydrogen unsubstituted methyl.

In a preferred embodiment

R_{w} is substituted or unsubstituted methyl, preferably unsubstituted methyl, while R_{w′} is hydrogen.

In a preferred embodiment

R_{w} and R_{w′} are both substituted or unsubstituted methyl, preferably unsubstituted methyl.

In a preferred embodiment of Formula (I),

R₁ is a substituted or unsubstituted pyridine, preferably a pyridine of formula

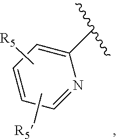

more preferably of formula

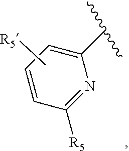

even more preferably of formula

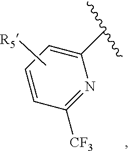

even more preferably of formula

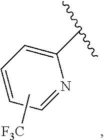

most preferably of formula

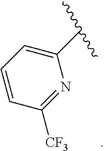 [SMILES: *c1cccc(C(F)(F)F)n1]

In a preferred embodiment for Formula (I′), (I²′), (I³′) or (l⁴′),

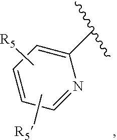

is selected from

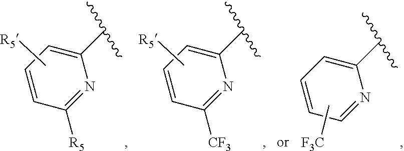

or preferably

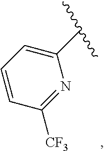 [SMILES: *c1cccc(C(F)(F)F)n1]

In a preferred embodiment for Formula (I′),

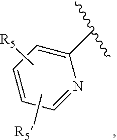

is selected from

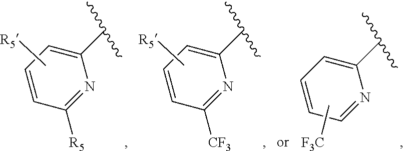

or preferably

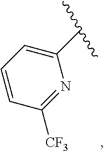

wherein R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR R¹¹R_{11′}, —OCH₂CH₂OR₁₁, —NR₁₁S(O)₂NR_{11′}R_{11″} and C(CH₃)₂OR₁₁;
and R₁₁, R_{11′} and R_{11″} are independently selected from hydrogen, unsubstituted C₁₋₆ alkyl, unsubstituted C₂₋₆ alkenyl and unsubstituted C₂₋₆ alkynyl.

In a preferred embodiment for Formula (I²′)

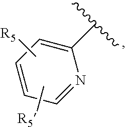

is selected from

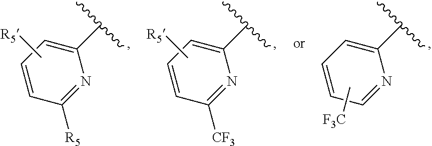

or preferably

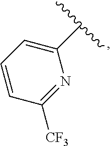

wherein R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
and R₁₁, R_{11′} and R_{11″} are independently selected from hydrogen, unsubstituted C₁₋₆ alkyl, unsubstituted C₂₋₆ alkenyl and unsubstituted C₂₋₆ alkynyl.

In a preferred embodiment for Formula (I³′)

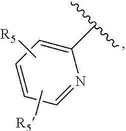

is selected from

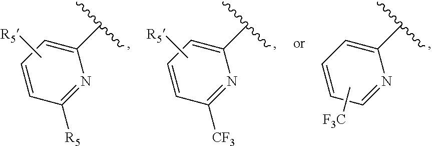

or preferably

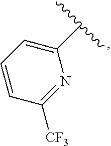

wherein R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
and R₁₁, R_{11′} and R_{11″} are independently selected from hydrogen, unsubstituted C₁₋₆ alkyl, unsubstituted C₂₋₆ alkenyl and unsubstituted C₂₋₆ alkynyl.

In a preferred embodiment for Formula (I⁴′)

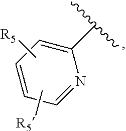

is selected from

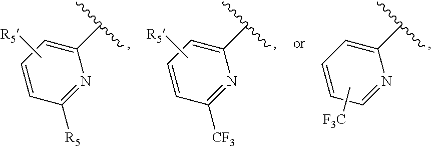

or preferably

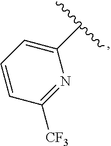

wherein R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
and R₁₁, R_{11′} and R_{11″} are independently selected from hydrogen, unsubstituted C₁₋₆ alkyl, unsubstituted C₂₋₆ alkenyl and unsubstituted C₂₋₆ alkynyl.

In a preferred embodiment

R₂ is hydrogen or a substituted or unsubstituted group selected from methyl, ethyl, isopropyl and phenyl, more preferably hydrogen or an unsubstituted group selected from methyl, ethyl, isopropyl and phenyl.

In a preferred embodiment

R₃ is substituted or unsubstituted ethyl, preferably unsubstituted ethyl.

In a preferred embodiment

R₄ is hydrogen.

In a preferred embodiment

R_{4′} is hydrogen.

In a preferred embodiment

R_{4″} is hydrogen.

In a preferred embodiment

R_{4′″} is hydrogen.

In a preferred embodiment

R₄ and R_{4′} are both hydrogen.

In a preferred embodiment

R_{4″} and R_{4′″} are both hydrogen.

In a preferred embodiment

R₄, R_{4′} R_{4″} and R_{4′″} are all hydrogen.

In a preferred embodiment

R₅ is —CF₃.

In a preferred embodiment

R₅ is —CF₃ on alpha position to the nitrogen of the pyridine moiety.

In a preferred embodiment

R_{5′} is hydrogen.

In a preferred embodiment

R₅ is —CF₃ while R_{5′} is hydrogen.

In a preferred embodiment

R₅ is —CF₃ on alpha position to the nitrogen of the pyridine moiety while R_{5′} is hydrogen.

In a preferred embodiment

Rₙ is hydrogen or substituted or unsubstituted methyl, preferably unsubstituted methyl.

In a preferred embodiment

R₁₂ is substituted or unsubstituted ethyl, preferably unsubstituted ethyl.

In an particular embodiment

the halogen is fluorine.

In an particular embodiment

the haloalkyl is —CF₃.

In a preferred further embodiment, the compounds of the general Formula (I) are selected from

| EX | STRUCTURE | Chemical name |
|---|---|---|
| 1 | 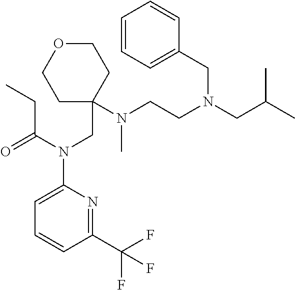 [SMILES: CCC(=O)N(CC1(N(C)CCN(Cc2ccccc2)CC(C)C)CCOCC1)c1cccc(C(F)(F)F)n1] | N-((4-((2- (benzyl(isobutyl)amino)ethyl)(methyl)amino)tetrahydro- 2H-pyran-4-yl)methyl)-N-(6-(trifluoromethyl)pyridin- 2-yl)propionamide |
| 2 | 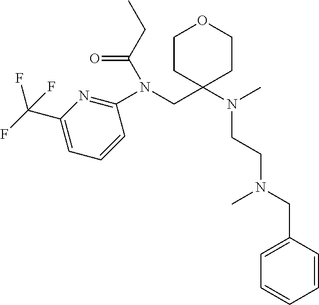 [SMILES: CCC(=O)N(CC1(N(C)CCN(C)Cc2ccccc2)CCOCC1)c1cccc(C(F)(F)F)n1] | N-((4-((2- (benzyl(methyl)amino)ethyl)(methyl)amino)tetrahydro- 2H-pyran-4-yl)methyl)-N-(6-(trifluoromethyl)pyridin- 2-yl)propionamide |
| 3 | 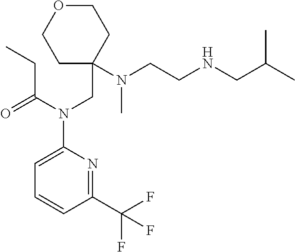 [SMILES: CCC(=O)N(CC1(N(C)CCNCC(C)C)CCOCC1)c1cccc(C(F)(F)F)n1] | N-((4-((2- (isobutylamino)ethyl)(methyl)amino)tetrahydro-2H- pyran-4-yl)methyl)-N-(6-(trifluoromethyl)pyridin-2- yl)propionamide |
| 4 | 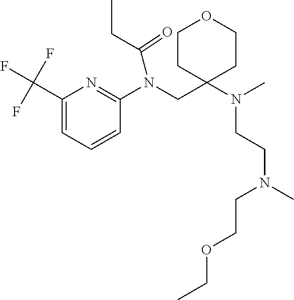 [SMILES: CCOCCN(C)CCN(C)C1(CN(C(=O)CC)c2cccc(C(F)(F)F)n2)CCOCC1] | N-((4-((2-((2-ethoxy- ethyl)(methyl)amino)ethyl)(methyl)amino)tetrahydro- 2H-pyran-4-yl)methyl)-N-(6- (trifluoromethyl)pyridin-2-yl)propionamide |
| 5 | 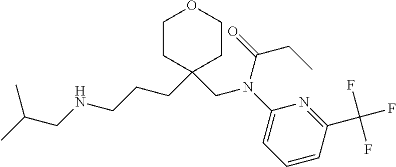 [SMILES: CCC(=O)N(CC1(CCCNCC(C)C)CCOCC1)c1cccc(C(F)(F)F)n1] | N-((4-(3-(isobutylamino)propyl)tetrahydro-2H-pyran- 4-yl)methyl)-N-(6-(trifluoromethyl)pyridin-2- yl)propionamide |
| 6 | 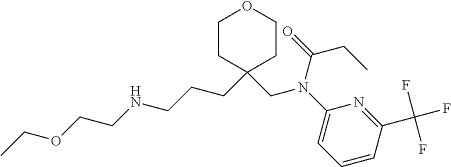 [SMILES: CCOCCNCCCC1(CN(C(=O)CC)c2cccc(C(F)(F)F)n2)CCOCC1] | N-((4-(3-((2-ethoxyethyl)amino)propyl)tetrahydro-2H- pyran-4-yl)methyl)-N-(6-(trifluoromethyl)pyridin-2- yl)propionamide |
| 7 | 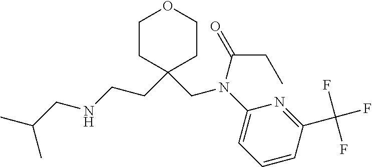 [SMILES: CCC(=O)N(CC1(CCNCC(C)C)CCOCC1)c1cccc(C(F)(F)F)n1] | N-((4-(2-(isobutylamino)ethyl)tetrahydro-2H-pyran-4- yl)methyl)-N-(6-(trifluoromethyl)pyridin-2- yl)propionamide |
| 8 | 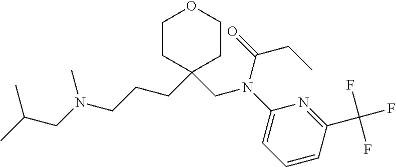 [SMILES: CCC(=O)N(CC1(CCCN(C)CC(C)C)CCOCC1)c1cccc(C(F)(F)F)n1] | N-((4-(3-(isobutyl(methyl)amino)propyl)tetrahydro-2H- pyran-4-yl)methyl)-N-(6-(trifluoromethyl)pyridin-2- yl)propionamide |
| 9 | 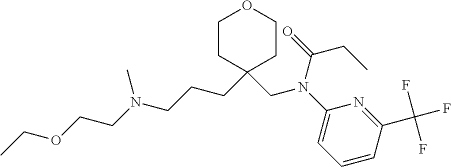 [SMILES: CCOCCN(C)CCCC1(CN(C(=O)CC)c2cccc(C(F)(F)F)n2)CCOCC1] | N-((4-(3-((2- ethoxyethyl)(methyl)amino)propyl)tetrahydro-2H- pyran-4-yl)methyl)-N-(6-(trifluoromethyl)pyridin-2- yl)propionamide |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
wherein said cycloalkyl, aryl or heterocyclyl in R₁ if substituted, is substituted with one or more substituent/s selected from halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
wherein the alkyl, alkenyl or alkynyl in R₁, if substituted, is substituted with one or more substituent/s selected from —OR₁₁, halogen, —CN, haloalkyl, haloalkoxy and —NR₁₁R_{11′};
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
wherein said cycloalkyl, aryl or heterocyclyl in R₂, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and C(CH₃)₂OR₁₂;
wherein the alkyl, alkenyl or alkynyl in R₂, if substituted, is substituted with one or more substituent/s selected from —OR₁₂, halogen, —CN, haloalkyl, haloalkoxy, —NR₁₂R_{12′};
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

the alkyl, alkenyl or alkynyl, other than those defined in R₁ or R₂, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

the aryl, heterocyclyl or cycloalkyl other than those defined in R₁ or R₂, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and —C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,
the cycloalkyl, aryl or heterocyclyl in R₁ if substituted, is substituted with one or more substituent/s selected from halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,
the cycloalkyl, aryl or heterocyclyl in R₁ if substituted, is substituted with haloalkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,

the alkyl, alkenyl or alkynyl in R₁, if substituted, is substituted with one or more substituent/s selected from OR₁₁, halogen, —CN, haloalkyl, haloalkoxy and —NR₁₁R_{11′};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₂ of any of the embodiments of the present invention,
the cycloalkyl, aryl or heterocyclyl in R₂, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and C(CH₃)₂OR₁₂;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₂ of any of the embodiments of the present invention,

the alkyl, alkenyl or alkynyl in R₂, if substituted, is substituted with one or more substituent/s selected from —OR₁₂, halogen, —CN, haloalkyl, haloalkoxy, —NR₁₂R_{12′};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₂ of any of the embodiments of the present invention,

the alkyl, alkenyl or alkynyl in R₂, if substituted, is substituted with —OR₁₂;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to alkyls other than those defined in R₁ or R₂ of any of the embodiments of the present invention,

the alkyl, alkenyl or alkynyl, other than those defined in R₁ or R₂, if substituted, is substituted with one or more substituent/s selected from —OR₁₃, halogen, —CN, haloalkyl, haloalkoxy and —NR₁₃R_{13′};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to the cycloalkyl, aryl or heterocyclyl other than those defined in R₁ or R₂ of any of the embodiments of the present invention,

the aryl, heterocyclyl or cycloalkyl other than those defined in R₁ or R₂, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and —C(CH₃)₂OR₁₄;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the halogen is fluorine, chlorine, iodine or bromine, preferably fluorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the haloalkyl is —CF₃;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the compound according to the invention of general Formula (I),

the haloalkoxy is —OCF₃;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

As this invention is aimed at providing a compound or a chemically related series of compounds which act as dual ligands of the σ₁ receptor and the μ-opioid receptor it is a very preferred embodiment in which the compounds are selected which act as dual ligands of the σ₁ receptor and the μ-opioid receptor and especially compounds which have a binding expressed as Kᵢ which is preferably <1000 nM for both receptors, more preferably <500 nM, even more preferably <100 nM.

In the following the phrase “compound of the invention” is used. This is to be understood as any compound according to the invention as described above according to general Formulae (I), (I′), (I²′), (I³′) or (I⁴′).

The compounds of the invention represented by the above described Formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

In general the processes are described below in the experimental part. The starting materials are commercially available or can be prepared by conventional methods.

A preferred aspect of the invention is also a process for the production of a compound according to Formula (I), following scheme 1.

A preferred aspect of the invention is a process for the production of a compound according to Formula (I), wherein R₁, R₂, R₃, R₄, R_{4′}, R_{4″}, R_{4″′}, R₅, R_{5′}, Rₙ, R_{w}, R_{w′}, m, n, p, q, Y and W are as defined in the description, following scheme 1.

For the sake of clarity the expression “a compound according to Formula (I), wherein R₁, etc. are as defined in the description” would (just like the expression “a compound of Formula (I) as defined in any one of claims e.g. 1 to 10” found in the claims) refer to “a compound according to Formula (I)”, wherein the definitions of the respective substituents R₁ etc. (also from the cited claims) are applied. In addition, this would also mean, though (especially in regards to the claims) that also one or more disclaimers defined in the description (or used in any of the cited claims like e.g. claim 1) would be applicable to define the respective compound. Thus, a disclaimer found in e.g. claim 1 would be also used to define the compound “of Formula (I) as defined in any one of the corresponding related claims e.g. 1 to 10”.

In all processes and uses described underneath and in scheme 1, the values of R₁, R₂, R₃, R₄, R_{4′}, R_{4″}, R_{4′″}, Rₙ, R_{w}, R_{w′}, m, n, p, q, Y and W are as defined in the description, L is a leaving group such as halogen or triflate and X is halogen.

A preferred embodiment of the invention is a process for the production of a compound according to Formula (I),
a) wherein n is 0, said process comprises the acylation of the NH group of compounds VII

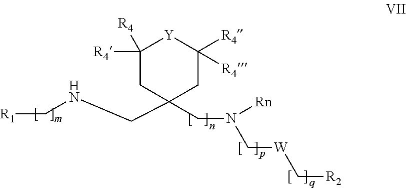

with an acyl halide of formula VIIIa or with an anhydride of formula VIIIb

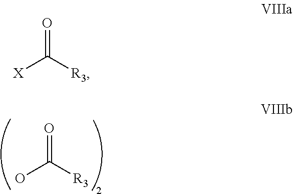

or
b) wherein n is 1, 2 or 3, said process comprises treating a compound of general formula XV

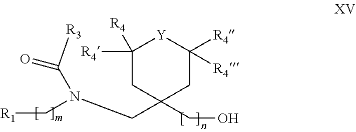

with trifluoromethanesulfonic anhydride, in the presence of a base, in a suitable solvent, such as dichloromethane, at a suitable temperature, preferably at −78° C., and subsequent reaction of the triflate intermediate with an amino derivative of formula III

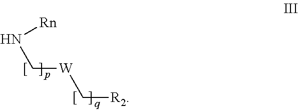

A preferred embodiment of the invention is a process for the production of a compound according to Formula (I), wherein n is 0,
said process comprises the acylation of the NH group of compounds VII

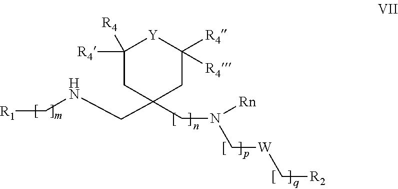

with an acyl halide of formula VIIIa or with an anhydride of formula VIIIb

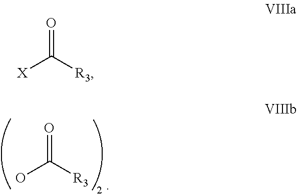

A preferred embodiment of the invention is a process for the production of a compound according to Formula (I), wherein n is 1, 2 or 3,

said process comprises treating a compound of general formula XV

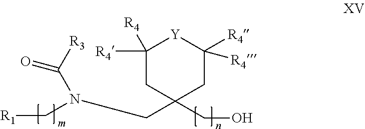

with trifluoromethanesulfonic anhydride, in the presence of a base, in a suitable solvent, such as dichloromethane, at a suitable temperature, preferably at −78° C., and subsequent reaction of the triflate intermediate with an amino derivative of formula III

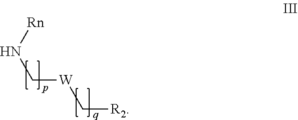

A preferred embodiment of the invention is a process, wherein n is 0, for the production of a compound according to Formula (IV) starting from a compound of Formula (II),

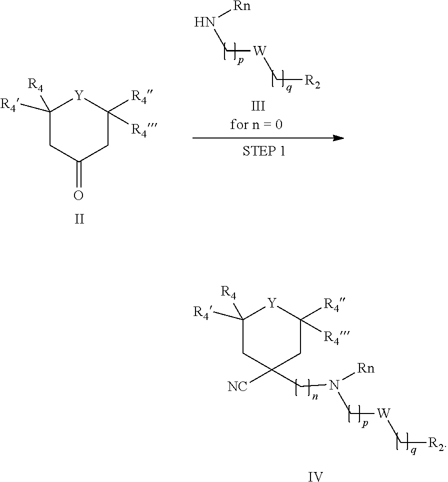

A preferred embodiment of the invention is a process, wherein n is 0, for the production of a compound according to Formula (V) starting from a compound of Formula (IV),

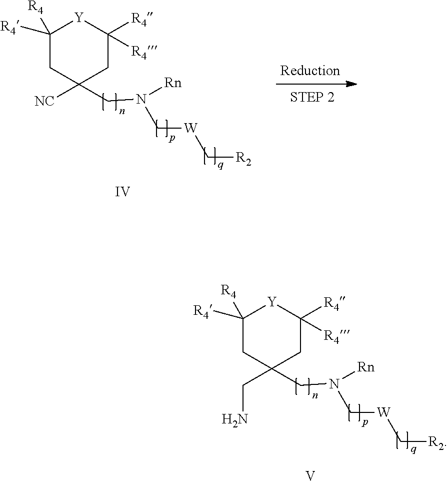

A preferred embodiment of the invention is a process, wherein n is 0, for the production of a compound according to Formula (VII) starting from a compound of Formula (V),

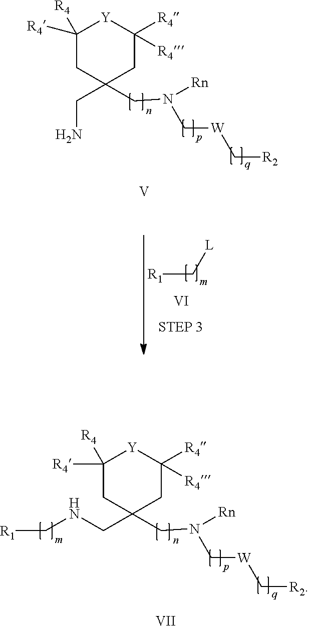

A preferred embodiment of the invention is a process, wherein n is 0, for the production of a compound according to Formula (I) starting from a compound of Formula (VII),

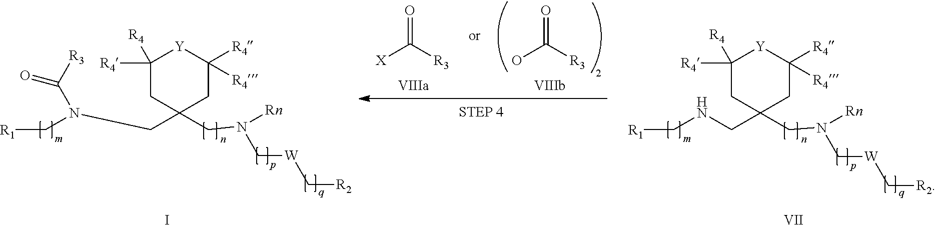

A preferred embodiment of the invention is a process, wherein n is 1, 2 or 3, for the production of a compound according to Formula (XI) starting from a compound of Formula (IX),

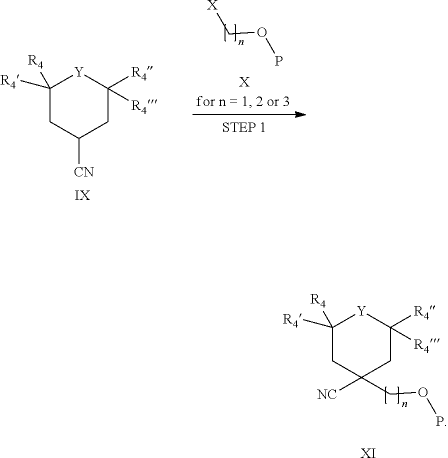

A preferred embodiment of the invention is a process, wherein n is 1, 2 or 3, for the production of a compound according to Formula (XII) starting from a compound of Formula (XI),

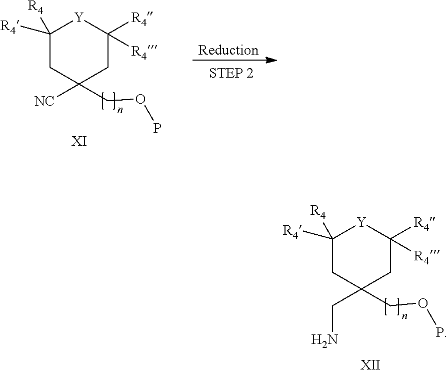

A preferred embodiment of the invention is a process, wherein n is 1, 2 or 3, for the production of a compound according to Formula (XIII) starting from a compound of Formula (XII),

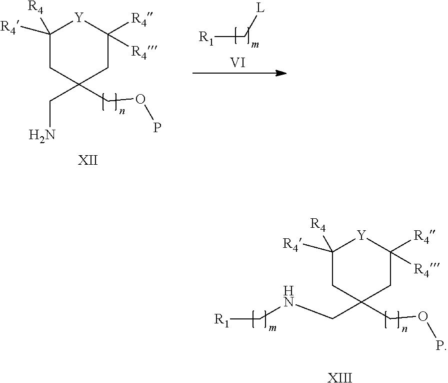

A preferred embodiment of the invention is a process, wherein n is 1, 2 or 3, for the production of a compound according to Formula (XIV) starting from a compound of Formula (XIII),

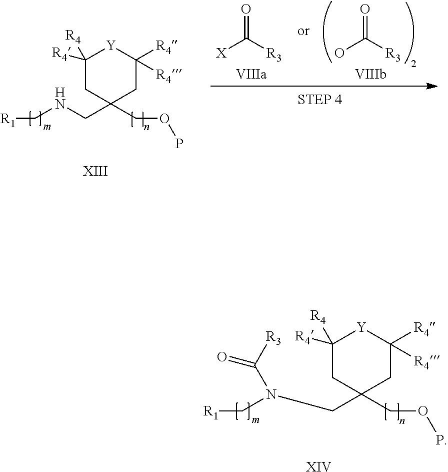

A preferred embodiment of the invention is a process, wherein n is 1, 2 or 3, for the production of a compound according to Formula (XV) starting from a compound of Formula (XIV),

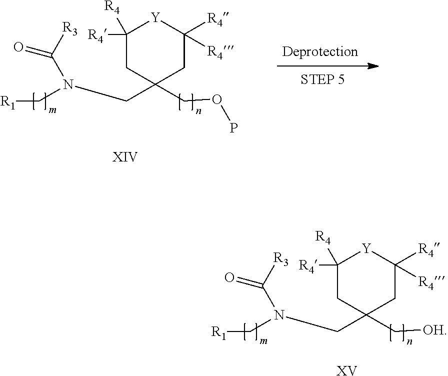

A preferred embodiment of the invention is a process, wherein n is 1, 2 or 3, for the production of a compound according to Formula (I) starting from a compound of Formula (XV),

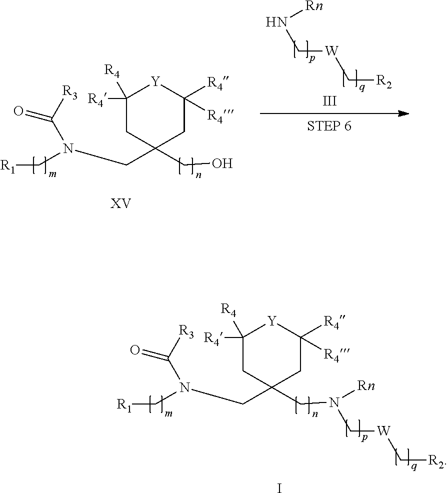

In another particular embodiment a compound of Formula (II),

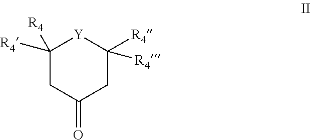

is used for the preparation of a compound of Formula (I).

In another particular embodiment a compound of Formula (III),

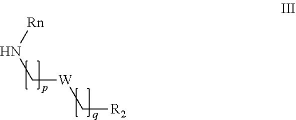

is used for the preparation of a compound of Formula (I).

In another particular embodiment a compound of Formula (IV),

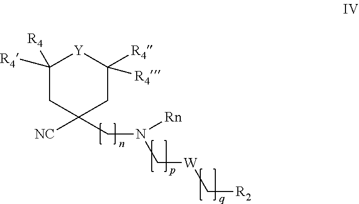

is used for the preparation of a compound of Formula (I).

In another particular embodiment a compound of Formula (V),

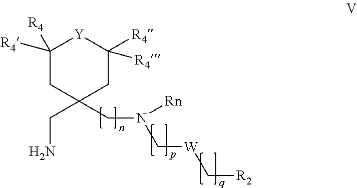

is used for the preparation of a compound of Formula (I).

In another particular embodiment a compound of Formula (VI),

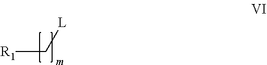

is used for the preparation of a compound of Formula (I).

In another particular embodiment a compound of Formula (VII),

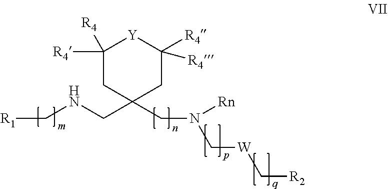

is used for the preparation of a compound of Formula (I).

In another particular embodiment a compound of Formula (VIIIa),

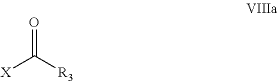

is used for the preparation of a compound of Formula (I).

In another particular embodiment a compound of Formula (VIIIb),

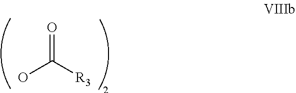

is used for the preparation of a compound of Formula (I).

In another particular embodiment a compound of Formula (IX),

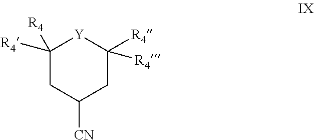

is used for the preparation of a compound of Formula (I).

In another particular embodiment a compound of Formula (X),

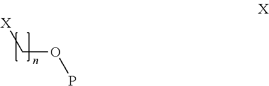

is used for the preparation of a compound of Formula (I).

In another particular embodiment a compound of Formula (XI),

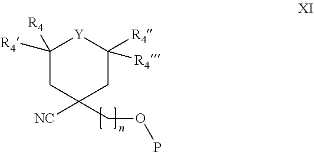

is used for the preparation of a compound of Formula (I).

In another particular embodiment a compound of Formula (XII),

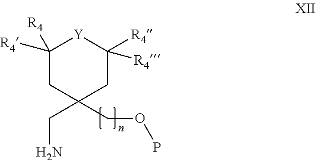

is used for the preparation of a compound of Formula (I).

In another particular embodiment a compound of Formula (XII),

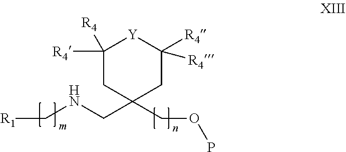

is used for the preparation of a compound of Formula (I).

In another particular embodiment a compound of Formula (XIV),

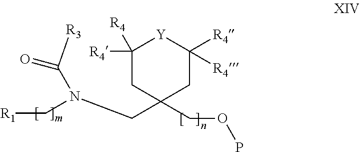

is used for the preparation of a compound of Formula (I).

In another particular embodiment a compound of Formula (XV),

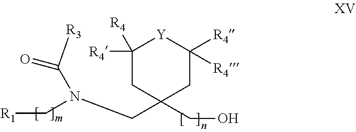

is used for the preparation of a compound of Formula (I).

In another particular embodiment there is a use of the compounds of Formula II, III, IV, V, VI, VII, VIIIa, VIIIb, IX, X, XI, XII, XIII, XIV or XV,

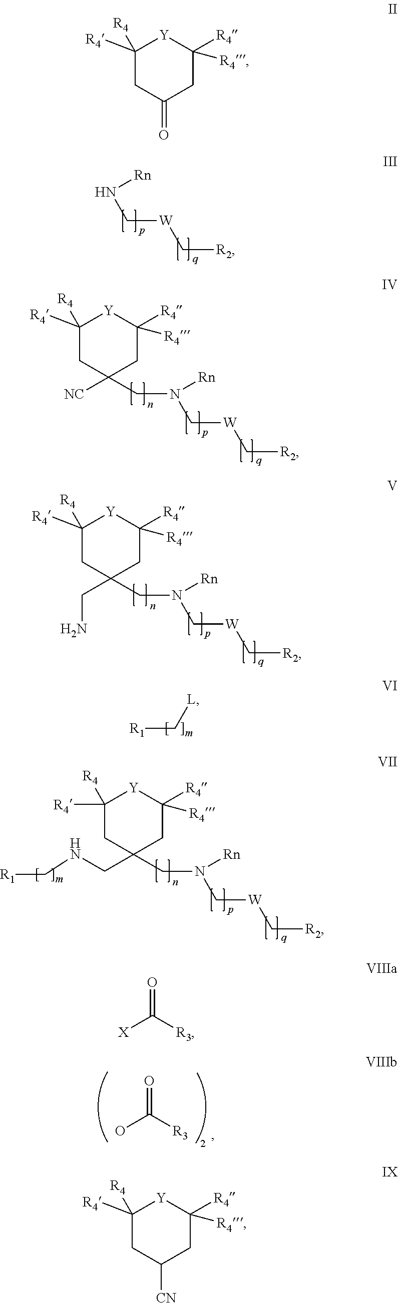

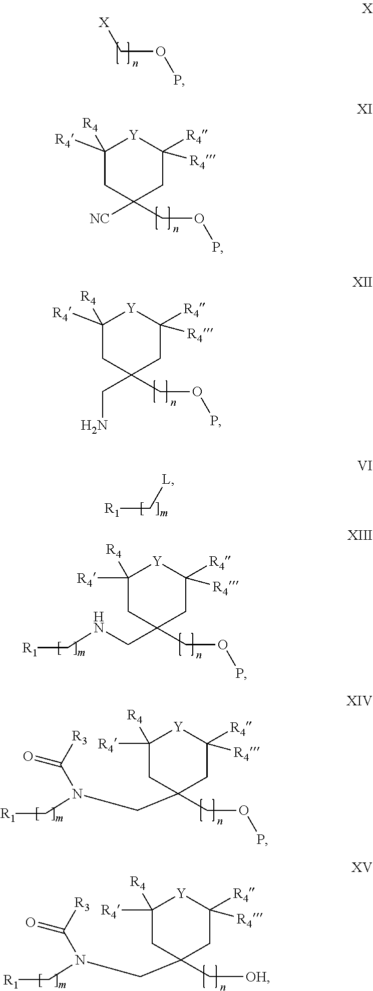

for the preparation of compounds of Formula (I).

Compounds of Formula II, III, IV, V, VI, VII, VIIIa, VIIIb, IX, X, XI, XII, XIII, XIV or XV,

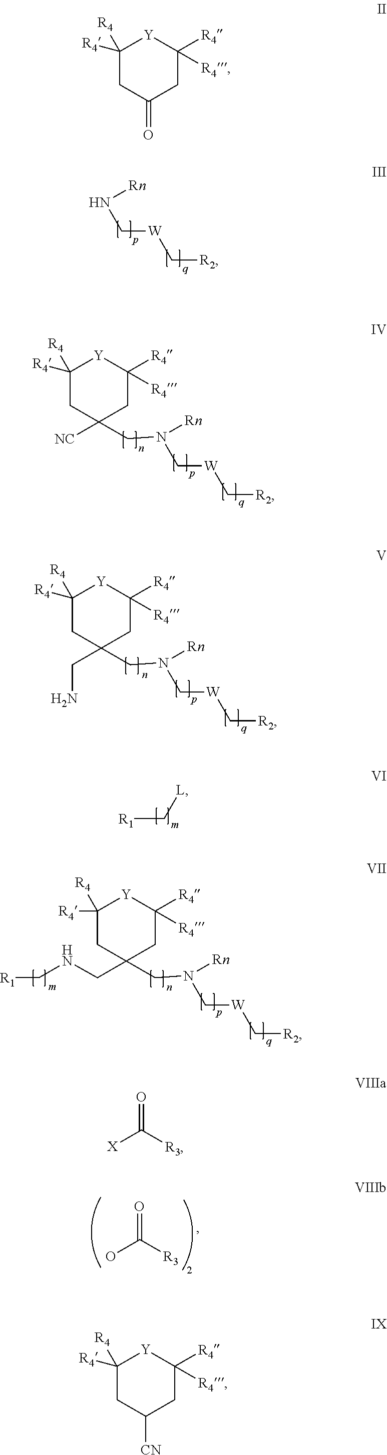

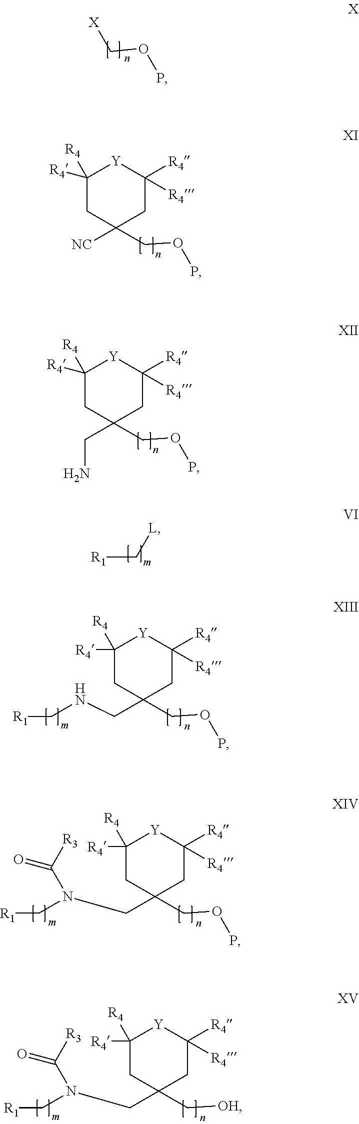

for use for the preparation of compounds of Formula (I).

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centres the compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention as described above according to general formula I or a pharmaceutically acceptable salt or stereoisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament for the treatment of pain. Preferably the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain.

In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment a therapeutically effective amount of a compound as above defined or a pharmaceutical composition thereof. Among the pain syndromes that can be treated are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

General Experimental Part (Methods and Equipment of the Synthesis and Analysis

A process is described in Scheme 1 for the preparation of compounds of general formula I, wherein R₁, R₂, R₃, R₄, R_{4′}, R_{4″}, R_{4″′}, Rn, W, m, p and q have the meanings defined above and n is 0.

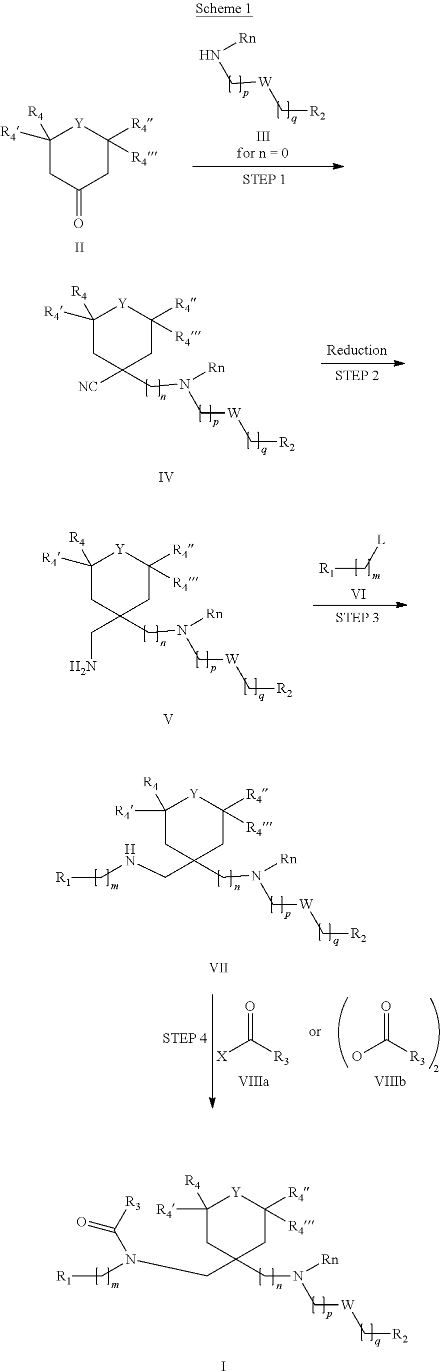

Where L is a leaving group such as halogen or triflate and X is halogen.

This process is carried out as described below:

Step 1: The compounds of general formula IV are prepared by Strecker reaction of ketone compounds of formula II with amino compounds of formula III using metal cyanide, preferably potassium cyanide, in the presence of an acid catalyst, in water at room temperature.

Step 2: The reduction of nitrile in compounds of formula IV renders compounds of general formula V. The reduction can be carried out in the presence of a suitable reducing agent such as lithium aluminium hydride, in a suitable solvent such as THF or diethylether, at a suitable temperature comprised between 0° C. and room temperature, preferably at room temperature. This reaction can be also effected with hydrogen at a pressure comprised between 1 and 10 bar, in a suitable solvent such as methanol or ethanol, in the presence of Raney nickel, at a suitable temperature comprised between room temperature and the reflux temperature.

Step 3: The compounds of general formula VII are prepared by reaction of a compound of general formula V with a compound of formula VI. When m=0 and R₁=aryl, the arylation reaction is carried out under catalytic conditions using a palladium or copper catalyst, in the presence of a suitable ligand and a suitable base, in a suitable solvent, and at a suitable temperature, preferably heating at the reflux temperature or in a microwave reactor. When using palladium catalysts, such as tris(dibenzylideneacetone)dipalladium or palladium diacetate, 4,5-bis(diphenylphosphino)-9,9-dimethylxanthene (XAMPHOS) or 2,2′-is(diphenylphosphino)-1,1′-binaphthyl (BINAP) are the preferred ligands, cessium carbonate or sodium tert-butoxide are used preferably as the base and 1,4-dioxane, toluene or tetrahydrofuran are the solvents of choice. When m=1-3 and/or R₁=alkyl, the alkylation reaction can be carried out in a suitable solvent, such as acetonitrile, dichloromethane, 1,4-dioxane or dimethylformamide, preferably in acetonitrile, in the presence of an inorganic base such as K₂CO₃ or Cs₂CO₃, or an organic base such as triethylamine or diisopropylethylamine, at a suitable temperature comprised between room temperature and the reflux temperature, or alternatively, the reactions can be carried out in a microwave reactor. Additionally, an activating agent, such as NaI, can be used.

Step 4: Compounds of general formula I are prepared by acylation of the NH group of compounds VII with an acyl halide of formula VIIIa or with an anhydride of formula VIIIb. The reaction is carried out in the presence of a suitable solvent, such as acetonitrile, dichloromethane, 1,4-dioxane, 1,2-dicloroethane, toluene or dimethylformamide, in the presence of an organic base such as triethylamine, pyridine or diisopropylethylamine, at a suitable temperature comprised between room temperature and the reflux temperature, or alternatively, the reactions can be carried out in a microwave reactor.

A process for the preparation of compounds of general formula I, wherein R₁, R₂, R₃, R₄, R_{4′}, R_{4″}, R_{4′″}, Rn, W, m, p and q have the meanings defined above, and n is 1, 2 or 3, is described in Scheme 2:

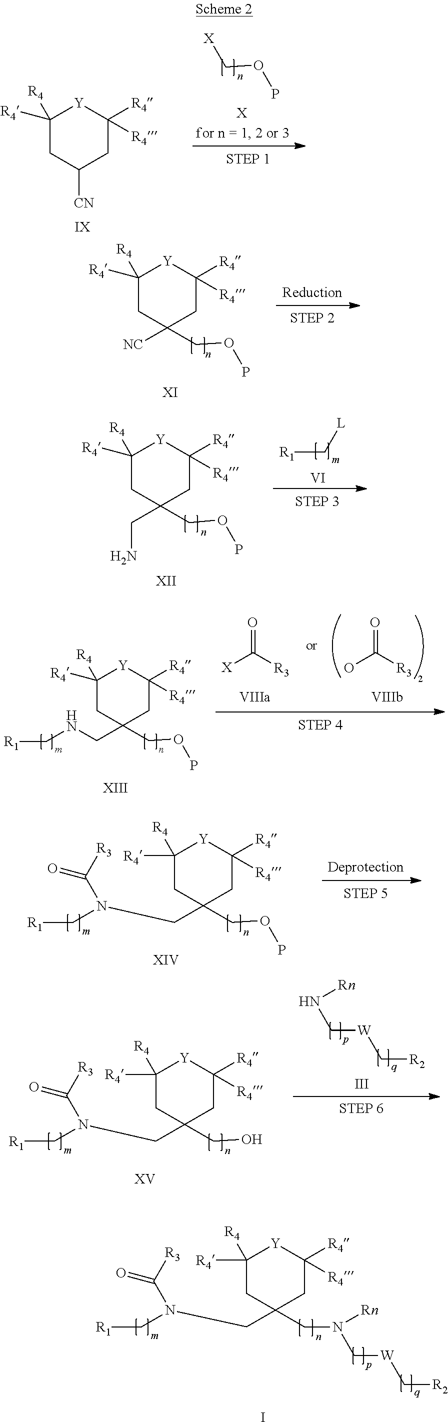

Where, L is a leaving group such as halogen or triflate and X is halogen.

This process is carried out as described below:

Step 1: A compound of formula XI is prepared by reaction of compound of formula IX with an appropriate alkylating reagent of formula X, in the presence of a strong base, such as LiHDMS, in a suitable solvent, preferably tetrahydrofuran, at a suitable temperature comprised between −78° C. and room temperature.

Step 2: The reduction of nitrile in compounds of formula XI renders compounds of general formula XII. The reduction can be carried out in similar conditions than those described in scheme 1, step 2.

Step 3: The synthesis of compounds of formula XIII, starting from compound of formula XII is performed in similar conditions than those described in scheme 1, step 3.

Step 4: Compounds of general formula XIV are prepared by acylation of the NH group of compounds XIII with an acyl halide of formula VIIIa or with an anhydride of formula VIIIb in similar conditions than those described in scheme 1, step 4.

Step 5: Deprotection of compounds of formula XIV to obtain compounds of formula XV is carried out, when P is benzyl, under hydrogenation conditions, preferably by treatment with ammonium formate as hydrogen source, in the presence of Pd, in a suitable solvent such as methanol or ethanol, at a suitable temperature comprised between room temperature and the reflux temperature, preferably at the reflux temperature.

Step 6: The compounds of general formula I are obtained by treating a compound of general formula XV with trifluoromethanesulfonic anhydride, in the presence of a base, in a suitable solvent, such as dichloromethane, at a suitable temperature, preferably at −78° C., and subsequent reaction of the triflate intermediate with an amino derivative of formula III. The alkylation reaction is carried out in in a suitable solvent, such as dimethylformamide, at a suitable temperature, preferably at room temperature.

The processes described in Schemes 1 and 2, represent the general routes for the preparation of compounds of formula I. Additionally, the order of the reactions may be changed and the functional groups present in any of the positions can be interconverted using reactions known to those skilled in the art.

Compounds of formula II, III, VI, VIIIa, VIIIb, IX and X where R₁, R₂, R₃, R₄, R_{4′}, R_{4″}, R_{4′″}, Rn, W, m, n, p and q have the meanings as defined above, are commercially available or can be prepared by conventional methods described in the bibliography.

### EXAMPLES

### Intermediates and Examples

The following abbreviations are used in the examples:

ACN: Acetonitrile

AcOEt: Ethyl acetate

Aq: Aqueous

BINAP: 2,2′-Bis(diphenylphosphino)-1,1′-binaphthyl

CH: Cyclohexane

DCM: Dichloromethane

DCE: 1,2-Dicloroethane

DIPEA: N,N-Diisopropylethylamine

DMF: Dimethylformamide

EtOH: Ethanol

Et₂O: Diethyl ether

Ex: Example

h: Hour/s

HPLC: High-performance liquid chromatography

INT: Intermediate

IPA: Isopropyl alcohol

MeOH: Methanol

MS: Mass spectrometry

Min: Minutes

Quant: Quantitative

Ret: Retention

rt: Room temperature

Sat: Saturated

THF: Tetrahydrofuran

Wt: Weight

The following methods were used to obtain the HPLC-MS data:

A: Column Acquity UPLC BEH C18 2.1×50 mm, 1.7 μm; flow rate 0.61 mL/min; A: NH₄HCO₃ 10 mM; B: ACN; Gradient: 0.3 min in 98% A, 98% A to 0% A in 2.7 min, 2 min in 0% A, 0% A to 98% A in 0.2 min, 0.55 min in 98% A

B: Column: Acquity BEH C18 2.1×50 mm 1.7 μm; flow rate 800 μl/min; A: NH₄HCO₃ 10 mM; B: ACN; Gradient: 0.3 min in 90% A, 90% A to 5% A in 2.7 min, 0.7 min in 5% A, 5% A to 90% A in 0.1 min, 1.2 min in 90% A

### Intermediate 1A. 4-((2-(Benzyl(isobutyl)amino)ethyl)(methyl)amino)tetrahydro-2H-pyran-4-carbonitrile

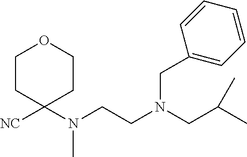 [SMILES: CC(C)CN(CCN(C)C1(C#N)CCOCC1)Cc1ccccc1]

In a 50 mL round bottomed flask, dihydro-2H-pyran-4(31)-one (0.45 g, 4.5 mmol) was dissolved in water (25 mL) along with sodium metabisulfite (0.32 g, 1.7 mmol). The mixture was stirred at rt for 1 h, then N¹-benzyl-N¹-isobutyl-N²-methylethane-1,2-diamine (0.75 g, 3.4 mmol) was added. The mixture was stirred for 2 h followed by the addition of potassium cyanide (0.35 g, 5.4 mmol). The colorless solution was extracted with ethyl acetate and the combined organic phases were washed with NaCl, dried over MgSO₄, filtered and concentrated to dryness to give the title compound as colorless oil (1.25 g, yield 90%)

¹H NMR (400 MHz, CDCl₃) δ ppm 0.42-7.25 (m, 4H), 7.27-7.16 (m, 1H), 3.94 (dt, J=12.4, 3.7 Hz, 2H), 3.60 (ddd, J=12.5, 11.5, 2.2 Hz, 2H), 3.56 (s, 2H), 2.57-2.46 (m, 4H), 2.24 (s, 3H), 2.20 (d, J=7.3 Hz, 2H), 1.97 (dq, J=13.5, 2.7 Hz, 2H), 1.86-1.69 (m, 1H), 1.63 (ddd, J=13.4, 11.6, 4.4 Hz, 2H), 0.89 (d, J=6.6 Hz, 6H)

This method was used for the preparation of intermediate 1B using suitable starting materials:

| | | Chemical | ¹H NMR (400 MHz |
|---|---|---|---|
| INT | Structure | name | Chloroform-d) δ ppm |
| 1B | 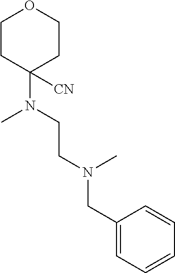 [SMILES: CN(CCN(C)C1(C#N)CCOCC1)Cc1ccccc1] | 4-((2- (Benzyl (methyl) amino)ethyl) (methyl) amino)tetra- hydro- 2H-pyran-4- carbonitrile | 7.36-7.27 (m, 4H), 7.30-7.22 (m, 1H), 3.98 (dt, J = 12.5, 3.6 Hz, 2H), 3.63 (td, J = 12.0, 2.1 Hz, 2H), 3.53 (s, 2H), 2.66 (dd, J = 8.5, 6.0 Hz, 2H), 2.52 (dd, J = 8.1, 5.7 Hz, 2H), 2.31 (s, 3H), 2.24 (s, 3H), 2.06 (dq, J = 13.5, 2.7 Hz, 2H), 1.72 (ddd, J = 13.3, 11.5, 4.4 Hz, 2H) |

### Intermediate 1C. 4-(3-(Benzyloxy)propyl)tetrahydro-2H-pyran-4-carbonitrile

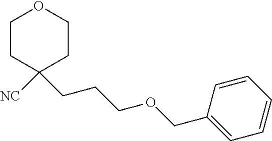 [SMILES: N#CC1(CCCOCc2ccccc2)CCOCC1]

To a solution of tetrahydro-2H-pyran-4-carbonitrile (2.15 g, 19.34 mmol) in dry THF (15 mL) cooled at −78° C., LiHDMS solution (1M in THF, 24.2 mL, 24.2 mmol) was added dropwise under nitrogen atmosphere and the mixture was stirred at this temperature for 1 h. Then, a solution of (3-iodopropoxy)methylbenzene (6.14 g, 22.2 mmol) in dry THF (10 mL) was added and the reaction mixture was allowed to reach rt and stirred overnight. The solvent was evaporated and the residue was diluted with water and Et₂O. The phases were separated and the aqueous phase was extracted several times with Et2O. The organic phases were combined and washed with water and brine, the solvent was evaporated and the residue thus obtained was purified by flash chromatography on silica gel, gradient CH to CH:AcOEt (80:20) to give the title compound (4.35 g, 87% yield).

HPLC-MS (Method B): Ret, 2.28 min; ESI+-MS m/z, 260.31 (M+H).

This method was used for the preparation of intermediate 1D using (2-iodoethoxy)methylbenzene as starting material:

| | | | | Ret | MS |
|---|---|---|---|---|---|
| INT | Structure | Chemical name | Method | (min) | (M + H) |
| 1D | 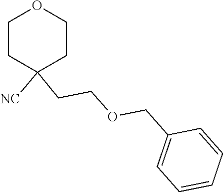 [SMILES: N#CC1(CCOCc2ccccc2)CCOCC1] | 4-(2- (benzyloxy)ethyl) tetrahydro-2H- pyran-4- carbonitrile | A | 1.78 | 246.1 |

### Intermediate 2A. N¹-(4-(Aminomethyl)tetrahydro-2H-pyran-4-yl)-N²-benzyl-N²-isobutyl-N¹-methylethane-1,2-diamine

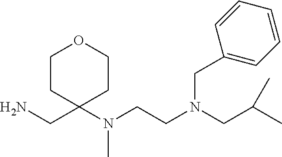 [SMILES: CC(C)CN(CCN(C)C1(CN)CCOCC1)Cc1ccccc1]

To a stirred fresh solution of lithium aluminium hydride (1M in Et₂O, 5.1 mL, 5 mmol) in Et₂O/THF (20/10 mL), a solution of 4-((2-(benzyl(isobutyl)amino)ethyl)(methyl)amino) tetrahydro-2H-pyran-4-carbonitrile (INT 1A, 0.85 g, 2.57 mmol) in Et₂O/THF (10/7 mL) was added at 0° C. under nitrogen atmosphere. The reaction was allowed to reach rt and stirred for 5 h. Then, NaOH 10% solution and AcOEt were added and the white suspension thus obtained was filtered. The organic layer was separated and washed with saturated aq NaCl solution. The organic layer was dried over Na₂SO₄, filtered and concentrated to give the title compound as colorless oil (0.75 g, 50% yield) that was used in the next step without further purification.

HPLC-MS (Method A): Ret, 1.92 min ESI⁺-MS m/z, 334 (M+1).

This method was used for the preparation of intermediates 2B-D using the corresponding intermediates 1B-D as starting materials:

| | | | | Ret | MS |
|---|---|---|---|---|---|
| INT | Structure | Chemical name | Method | (min) | (M + H) |
| 2B | 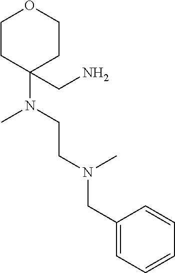 [SMILES: CN(CCN(C)C1(CN)CCOCC1)Cc1ccccc1] | N¹-(4-(amino- methyl)tetrahydro- 2H-pyran-4- yl)-N²-benzyl- N¹,N²- dimethylethane- 1,2-diamine | A | 1.33 | 292.2 |
| 2C | 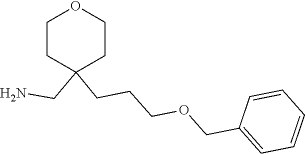 [SMILES: NCC1(CCCOCc2ccccc2)CCOCC1] | (4-(3- (benzyloxy)propyl) tetrahydro-2H- pyran-4- yl)methanamine | A | 1.32 | 264.2 |
| 2D | 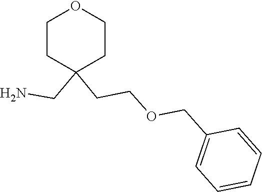 [SMILES: NCC1(CCOCc2ccccc2)CCOCC1] | (4-(2- (benzyloxy)ethyl) tetrahydro-2H- pyran-4- yl)methanamine | A | 1.21 | 250.1 |

### Intermediate 3A. N¹-Benzyl-N¹-isobutyl-N²-methyl-N²-(4-(((6-(trifluoromethyl)pyridin-2-yl)amino)methyl)tetrahydro-2H-pyran-4-yl)ethane-1,2-diamine

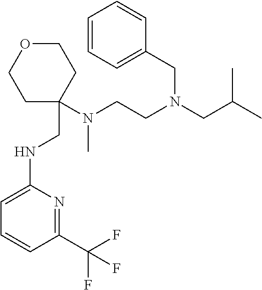 [SMILES: CC(C)CN(CCN(C)C1(CNc2cccc(C(F)(F)F)n2)CCOCC1)Cc1ccccc1]

N¹-(4-(Aminomethyl)tetrahydro-2H-pyran-4-yl)-N²-benzyl-N²-isobutyl-N¹-methylethane-1,2-diamine (INT 2A, 0.76 g, 2.3 mmol), 2-bromo-6-(trifluoromethyl)pyridine (0.5 g, 2.3 mmol), Pd₂(dba)₃ (0.21 g, 0.23 mmol), BINAP (0.17 g, 0.27 mmol) and ^{t}BuOK (0.51 g, 4.6 mmol) were charged in a Schlenk tube, purged under nitrogen, and dissolved in anhydrous THF (35 mL). The reaction mixture was stirred at 55-60° C. for 6 h. The solvent was evaporated and the crude product thus obtained was diluted with Et₂O (also a minimum of EtOAc) and a 2 N HCl solution. The layers were separated and the organic phase was extracted with more 2 N HCl solution. The aqueous phase was washed with AcOEt and basified with 10% NaOH up to pH 12. The aqueous phase was extracted with AcOEt and the combined organic phases were dried over Na₂SO₄, filtered and concentrated to give brown oil. This crude product was purified by flash chromatography on silica gel, gradient CH:AcOEt, from (100:0) to (50:50), to give the title compound as a colorless oil (0.43 g, 40% yield).

HPLC-MS (Method A): Ret, 2.98 min; ESI⁺-MS m/z, 479.3 (M+1).

This method was used for the preparation of intermediates 3B-D using the corresponding intermediates 2 as starting materials:

| | | | | Ret | MS |
|---|---|---|---|---|---|
| INT | Structure | Chemical name | Method | (min) | (M + H) |
| 3B | 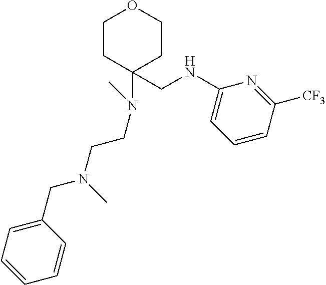 [SMILES: CN(CCN(C)C1(CNc2cccc(C(F)(F)F)n2)CCOCC1)Cc1ccccc1] | N¹-Benzyl-N¹,N²- dimethyl-N²-(4-(((6- (trifluoromethyl) pyridin-2- yl)amino)methyl) tetrahydro-2H-pyran- 4-yl)ethane-1,2- diamine | A | 2.43 | 437.4 |
| 3C | 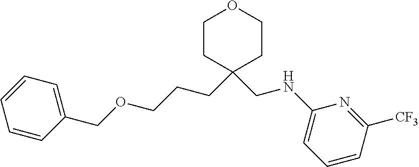 [SMILES: FC(F)(F)c1cccc(NCC2(CCCOCc3ccccc3)CCOCC2)n1] | N-((4-(3- (benzyloxy)propyl) tetrahydro-2H-pyran- 4-yl)methyl)-6- (trifluoromethyl) pyridin-2-amine | A | 2.40 | 409.2 |
| 3D | 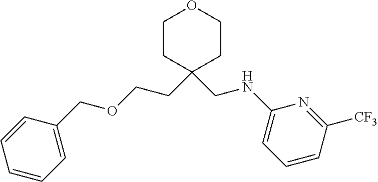 [SMILES: FC(F)(F)c1cccc(NCC2(CCOCc3ccccc3)CCOCC2)n1] | N-((4-(2- (benzyloxy)ethyl) tetrahydro-2H-pyran- 4-yl)methyl)-6- (trifluoromethyl) pyridin-2-amine | A | 2.89 | 395.4 |

### Intermediate 4A. N-((4-(3-(Benzyloxy)propyl)tetrahydro-2H-pyran-4-yl)methyl)-N-(6-(trifluoromethyl)pyridin-2-yl)propionamide

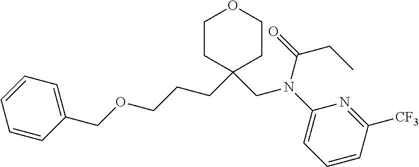 [SMILES: CCC(=O)N(CC1(CCCOCc2ccccc2)CCOCC1)c1cccc(C(F)(F)F)n1]

Propionyl chloride (1.7 mL, 19.8 mmol) was added to a solution of N-((4-(3-(benzyloxy)propyl)tetrahydro-2H-pyran-4-yl)methyl)-6-(trifluoromethyl)pyridin-2-amine (INT 3C, 2.13 g, 4.95 mmol), DIPEA (4.3 mL, 24.8 mmol) and DMAP (0.6 g, 5 mmol) in DCE (240 mL) under nitrogen atmosphere and the mixture was heated at 85° C. Additional propionyl chloride and DIPEA were added along 3 days until complete conversion was achieved (as monitored by TLC). The solvent was removed and the residue thus obtained was diluted with AcOEt and washed with NaHCO₃. The organic layer was dried over Na₂SO₄, filtered and concentrated to dryness. The crude thus obtained was purified by flash chromatography on silica gel, eluents CH/AcOEt, gradient from (100:0) to (40:60), to give the title compound (0.590 g, yield 26%).

HPLC-MS (Method A): Ret, 2.32 min; ESI⁺-MS m/z, 465.3 (M+1).

This method was used for the preparation of intermediate 4B using intermediate 3D as starting material:

| | | | | Ret | MS |
|---|---|---|---|---|---|
| INT | Structure | Chemical name | Method | (min) | (M + H) |
| 4B | 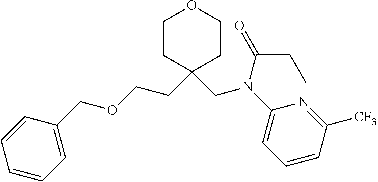 [SMILES: CCC(=O)N(CC1(CCOCc2ccccc2)CCOCC1)c1cccc(C(F)(F)F)n1] | N-((4-(2- (benzyloxy)ethyl) tetrahydro-2H- pyran-4- yl)methyl)-N-(6- (trifluoromethyl) pyridin-2- yl)propionamide | A | 2.25 | 451.2 |

### Intermediate 5A. N-((4-(3-Hydroxypropyl)tetrahydro-2H-pyran-4-yl)methyl)-N-(6-(trifluoromethyl)pyridin-2-yl)propionamide

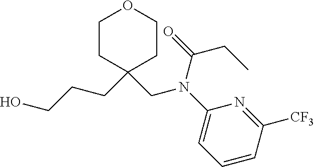 [SMILES: CCC(=O)N(CC1(CCCO)CCOCC1)c1cccc(C(F)(F)F)n1]

N-((4-(3-(benzyloxy)propyl)tetrahydro-2H-pyran-4-yl)methyl)-N-(6-(trifluoromethyl)pyridin-2-yl)propionamide (INT 4A, 0.55 g, 0.956 mmol) was dissolved in EtOH (30 mL) and ammonium formate (0.301 g, 4.8 mmol) and Pd/C (0.508 g, 10% Wt) were added in a sealed tube. The suspension was stirred under N₂ atmosphere for 3 days at 85 C. The reaction mixture was filtered through a Millipore system and the filtrate was washed with MeOH and concentrated, to give the title compound (0.36 g, quant yield) that was used in the following step without further purification.

HPLC-MS (Method A): Ret, 1.56 min; ESI⁺-MS m/z, 375.2 (M+1).

This method was used for the preparation of the following intermediate using the corresponding starting product:

| | | | | Ret | MS |
|---|---|---|---|---|---|
| INT | Structure | Chemical name | Method | (min) | (M + H) |
| 5B | 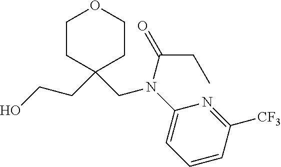 [SMILES: CCC(=O)N(CC1(CCO)CCOCC1)c1cccc(C(F)(F)F)n1] | N-((4-(2- hydroxyethyl)tetra- hydro-2H-pyran-4- yl)methyl)-N-(6- (trifluoromethyl) pyridin-2- yl)propionamide | A | 1.52 | 361.2 |

### Example 1

### N-((4-((2-(Benzyl(isobutyl)amino)ethyl)(methyl)amino)tetrahydro-2H-pyran-4-yl)methyl)-N-(6-(trifluoromethyl)pyridin-2-yl)propionamide

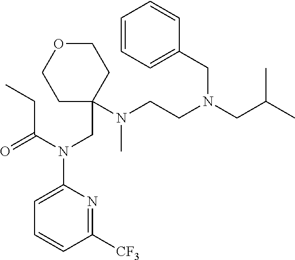 [SMILES: CCC(=O)N(CC1(N(C)CCN(Cc2ccccc2)CC(C)C)CCOCC1)c1cccc(C(F)(F)F)n1]

Propionyl chloride (200 μL, 2.3 mmol) was added to a solution of N¹-benzyl-N¹-isobutyl-N²-methyl-N²-(4-(((6-(trifluoromethyl)pyridin-2-yl)amino)methyl)tetrahydro-2H-pyran-4-yl)ethane-1,2-diamine (INT 3A, 0.37 g, 0.77 mmol) and DIPEA (500 μL, 3 mmol) in DCE (24 mL) in a process vial under nitrogen atmosphere. The reaction mixture was heated under microwave irradiation for 18 h at 80° C. The same amount of propionyl chloride and DIPEA were added and the mixture submitted to a second MW cycle. Then, the mixture was washed twice with water and the organic layer was dried with Na₂SO₄, filtered and concentrated to dryness. The crude product thus obtained was purified by flash chromatography on silica gel, eluents CH/AcOEt, gradient from (100:0) to (80:20), to give the title compound (0.314 mg, yield 75%).

HPLC-MS (Method A): Ret, 2.96 min; ESI+-MS m/z, 535.4 (M+1).

This method was used for the preparation of example 2 using intermediate 3B as starting material:

| | | | | Ret | MS |
|---|---|---|---|---|---|
| EX | Structure | Chemical name | Method | (min) | (M + H) |
| 2 | 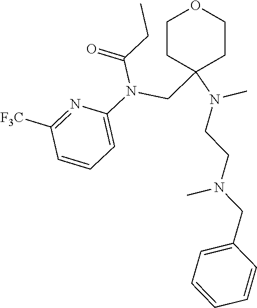 [SMILES: CCC(=O)N(CC1(N(C)CCN(C)Cc2ccccc2)CCOCC1)c1cccc(C(F)(F)F)n1] | N-((4-((2- (benzyl(methyl) amino)ethyl)(methyl) amino)tetrahydro- 2H-pyran-4- yl)methyl)-N-(6- (trifluoromethyl) pyridin-2- yl)propionamide | A | 2.34 | 493.5 |

### Example 3

### N-((4-((2-(Isobutylamino)ethyl)(methyl)amino)tetrahydro-2H-pyran-4-yl)methyl)-N-(6-(trifluoromethyl)pyridin-2-yl)propionamide

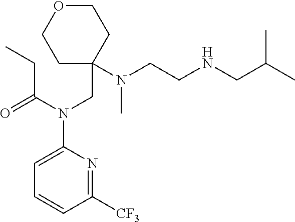 [SMILES: CCC(=O)N(CC1(N(C)CCNCC(C)C)CCOCC1)c1cccc(C(F)(F)F)n1]

N-((4-((2-(Benzyl(isobutyl)amino)ethyl)(methyl)amino)tetrahydro-2H-pyran-4-yl)methyl)-N-(6-(trifluoromethyl)pyridin-2-yl)propionamide (Example 1, 200 mg, 0.34 mmol) was dissolved in IPA (26 mL) and 3 drops of 6 N HCl and Pd/C (10% Wt, 75 mg, 0.70 mmol) were added. The suspension was stirred under 1 bar of H₂ at rt overnight. The reaction mixture was filtered through a Millipore system and the filtrate was washed with MeOH and concentrated. The crude product thus obtained was purified by flash chromatography on neutral alumina, eluents DCM:MeOH, gradient from (100:0) to (90:10), to give the title compound (60 mg, yield 37%).

HPLC-MS (Method A): Ret, 1.76 min; ESI⁺-MS m/z, 445.3 (M+1).

### Example 4

### N-((4-((2-((2-Ethoxyethyl)(methyl)amino)ethyl)(methyl)amino)tetrahydro-2H-pyran-4-yl)methyl)-N-(6-(trifluoromethyl)pyridin-2-yl)propionamide

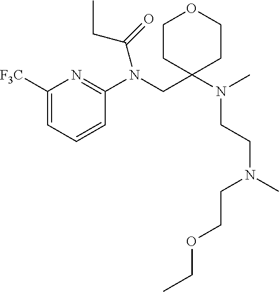 [SMILES: CCOCCN(C)CCN(C)C1(CN(C(=O)CC)c2cccc(C(F)(F)F)n2)CCOCC1]

### a) N-((4-(Methyl(2-(methylamino)ethyl)amino)tetrahydro-2H-pyran-4-yl)methyl)-N-(6-(trifluoromethyl)pyridin-2-yl)propionamide

N-((4-((2-(benzyl(methyl)amino)ethyl)(methyl)amino)tetrahydro-2H-pyran-4-yl)methyl)-N-(6-(trifluoromethyl)pyridin-2-yl)propionamide (Ex 2, 69 mg, 140 mmol) was dissolved in MeOH (4 mL) and ammonium formate (40 mg, 0.63 mmol) and Pd/C (15 mg, 10% Wt) were added. The suspension was stirred under N₂ atmosphere for 3 h at 65° C. The reaction mixture was filtered through a Millipore system and the filtrate was washed with MeOH and concentrated, to give the title compound (56 mg, quant yield), that was used in the following step without further purification.

HPLC-MS (Method A): Ret, 1.4 min; ESI+-MS m/z, 403.4 (M+1).

### b) Title Compound

1-Bromo-2-ethoxyethane (48 μL, 0.42 mmol) was added to a solution of the compound obtained in step a) (56 mg, 0.14 mmol) and K₂CO₃ (58 mg, 0.42 mmol) in ACN (6 mL). The reaction mixture was stirred at 70° C. for 2 days and then it was cooled down to rt. AcOEt (10 mL) and sat aq NaHCO₃ solution (10 mL) were added and the phases were separated. The organic layer was dried over Na₂SO₄, filtered and concentrated. The crude residue thus obtained was purified by preparative HPLC (Column X-Bridge C18, ACN: NH₄HCO₃ 10 mM from (2:98 to 95-5), flow 20 ml/min, rt, to give the title compound as an oil (5 mg, yield 8%).

HPLC-MS (Method A): Ret, 1.86 min; ESI⁺-MS m/z, 475.4 (M+1).

### Example 5

### N-((4-(3-(Isobutylamino)propyl)tetrahydro-2H-pyran-4-yl)methyl)-N-(6-(trifluoromethyl)pyridin-2-yl)propionamide

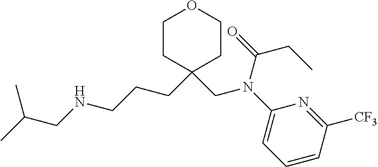 [SMILES: CCC(=O)N(CC1(CCCNCC(C)C)CCOCC1)c1cccc(C(F)(F)F)n1]

To a stirring solution of N-((4-(3-hydroxypropyl)tetrahydro-2H-pyran-4-yl)methyl)-N-(6-(trifluoromethyl)pyridin-2-yl)propionamide (INT 5A, 220 mg, 0.59 mmol) in anh DCM (41 mL), cooled at −78° C., 2,6-lutidine (0.32 mL, 2.73 mmol) followed by trifluoromethanesulfonic anhydride (1M solution in DCM, 1.17 mL, 1.17 mmol) were added under aN₂ atmosphere. The mixture was stirred at −78° C. for 2 h and then a solution of 2-methylpropan-1-amine (0.23 mL, 2.35 mmol) in dry DMF (5 mL) was added dropwise. The mixture was allowed to reach rt and stirred overnight. The solvent was removed and the residue was diluted with AcOEt and washed with sat aq NaHCO₃ solution. The organic layer was dried over Na₂SO₄, filtered and concentrated to dryness. The crude product thus obtained was purified by flash chromatography on silica gel, eluents DCM/DCM:NH₃ (0.25 M in EtOH) (6:4) gradient from 15% to 77%, to give the title compound (102 mg, yield 40%).

HPLC-MS (Method A): Ret, 1.6 min; ESI+-MS m/z, 430.3 (M+H).

This method was used for the preparation of examples 6-7 using suitable starting products:

| | | | | Ret | MS |
|---|---|---|---|---|---|
| Ex | Structure | Chemical name | Method | (min) | (M + H) |
| 6 | 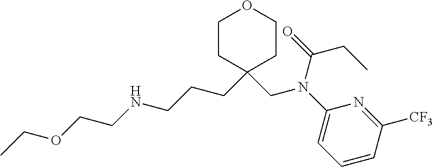 [SMILES: CCOCCNCCCC1(CN(C(=O)CC)c2cccc(C(F)(F)F)n2)CCOCC1] | N-((4-(3-((2- ethoxyethyl)amino) propyl)tetrahydro-2H- pyran-4-yl)methyl)- N-(6-(trifluoro- methyl)pyridin-2- yl)propionamide | A | 1.56 | 446.3 |
| 7 | 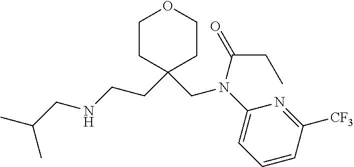 [SMILES: CCC(=O)N(CC1(CCNCC(C)C)CCOCC1)c1cccc(C(F)(F)F)n1] | N-((4-(2- (isobutylamino)ethyl) tetrahydro-2H- pyran-4-yl)methyl)- N-(6-(trifluoro- methyl)pyridin-2- yl)propionamide | A | 1.62 | 416.4 |

### Example 8

### N-((4-(3-(isobutyl(methyl)amino)propyl)tetrahydro-2H-pyran-4-yl)methyl)-N-(6-(trifluoromethyl)pyridin-2-yl)propionamide

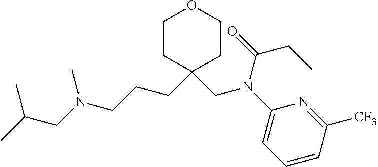 [SMILES: CCC(=O)N(CC1(CCCN(C)CC(C)C)CCOCC1)c1cccc(C(F)(F)F)n1]

N-((4-(3-(Isobutylamino)propyl)tetrahydro-2H-pyran-4-yl)methyl)-N-(6-(trifluoromethyl)pyridin-2-yl)propionamide (Ex 5, 38 mg, 0.09 mmol) was dissolved in formic acid (0.33 mL, 8.8 mmol) and formaldehyde (37% aq solution, 0.7 mL, 8.8 mmol) and heated at 90° C. in a sealed tube overnight. The mixture was partitioned between AcOEt and sat aq NaHCO₃ solution. The organic layer was dried over Na₂SO₄, filtered and concentrated. The crude residue was purified by flash chromatography on silica gel, gradient DCM:MeOH from (95:5) to (60:40), to give the title compound (40 mg, yield 100%).

HPLC-MS (Method A): Ret, 2.44 min; ESI⁺-MS m/z, 444.5 (M+1).

This method was used for the preparation of example 9 using the compound obtained in example 6 as starting material.

| | | | | Ret | MS |
|---|---|---|---|---|---|
| EX | Structure | Chemical name | Method | (min) | (M + H) |
| 9 | 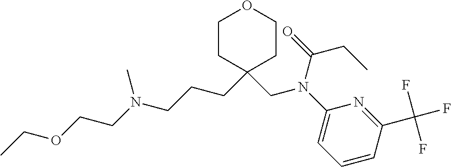 [SMILES: CCOCCN(C)CCCC1(CN(C(=O)CC)c2cccc(C(F)(F)F)n2)CCOCC1] | N-((4-(3-((2- ethoxyethyl)(methyl) amino)propyl)tetra- hydro-2H-pyran-4- yl)methyl)-N-(6- (trifluoromethyl) pyridin-2- yl)propionamide | B | 2.18 | 460.5 |

Table of Examples with Binding to the μ-Opioid Receptor and the σ₁-Receptor:

Biological Activity

Pharmacological Study

Human σ₁ Receptor Radioligand Assay

To investigate binding properties of test compounds to human σ₁ receptor, transfected HEK-293 membranes and [³H](+)-pentazocine (Perkin Elmer, NET-1056), as the radioligand, were used. The assay was carried out with 7 μg of membrane suspension, 5 nM of [³H](+)-pentazocine in either absence or presence of either buffer or 10 μM Haloperidol for total and non-specific binding, respectively. Binding buffer contained Tris-HCl 50 mM at pH 8. Plates were incubated at 37° C. for 120 minutes. After the incubation period, the reaction mix was then transferred to MultiScreen HTS, FC plates (Millipore), filtered and plates were washed 3 times with ice-cold 10 mM TrisHCL (pH7.4). Filters were dried and counted at approximately 40% efficiency in a MicroBeta scintillation counter (Perkin-Elmer) using EcoScint liquid scintillation cocktail.

Preferably, transfected HEK-293 membranes (7 μg) were incubated with 5 nM of [³H](+)-pentazocine in assay buffer containing Tris-HCl 50 mM at pH 8. NBS (non-specific binding) was measured by adding 10 μM Haloperidol. The binding of the test compound was measured at five different concentrations. Plates were incubated at 37° C. for 120 minutes. After the incubation period, the reaction mix was then transferred to MultiScreen HTS, FC plates (Millipore), filtered and plates were washed 3 times with ice-cold 10 mM TrisHCL (pH7.4). Filters were dried and counted at approximately 40% efficiency in a MicroBeta scintillation counter (Perkin-Elmer) using EcoScint liquid scintillation cocktail.

Human μ-Opioid Receptor Radioligand Assay

To investigate binding properties of test compounds to human μ-opioid receptor, transfected CHO-K1 cell membranes and [³H]-DAMGO (Perkin Elmer, ES-542-C), as the radioligand, were used. The assay was carried out with 20 μg of membrane suspension, 1 nM of [³H]-DAMGO in either absence or presence of either buffer or 10 μM Naloxone for total and non-specific binding, respectively. Binding buffer contained Tris-HCl 50 mM, MgCl2 5 mM at pH 7.4. Plates were incubated at 27° C. for 60 minutes. After the incubation period, the reaction mix was then transferred to MultiScreen HTS, FC plates (Millipore), filtered and plates were washed 3 times with ice-cold 10 mM TrisHCL (pH 7.4). Filters were dried and counted at approximately 40% efficiency in a MicroBeta scintillation counter (Perkin-Elmer) using EcoScint liquid scintillation cocktail.

Preferably, transfected CHO-K1 cell membranes (20 μg) were incubated with 1 nM of [³H]-DAMGO in assay buffer containing Tris-HCl 50 mM, MgCl2 5 mM at pH 7.4. NBS (non-specific binding) was measured by adding 10 μM Naloxone. The binding of the test compound was measured at five different concentrations. Plates were incubated at 27° C. for 60 minutes. After the incubation period, the reaction mix was then transferred to MultiScreen HTS, FC plates (Millipore), filtered and plates were washed 3 times with ice-cold 10 mM TrisHCL (pH 7.4). Filters were dried and counted at approximately 40% efficiency in a MicroBeta scintillation counter (Perkin-Elmer) using EcoScint liquid scintillation cocktail.

Results:

As this invention is aimed at providing a compound or a chemically related series of compounds which act as dual ligands of the σ₁ receptor and the μ-opioid receptor it is a very preferred embodiment in which the compounds are selected which act as dual ligands of the σ₁ receptor and the μ-opioid receptor and especially compounds which have a binding expressed as Kᵢ which is preferably <1000 nM for both receptors, more preferably <500 nM, even more preferably <100 nM.

The following scale has been adopted for representing the binding to the σ₁ receptor and the μ-opioid receptor expressed as Kᵢ:
+ Both Kᵢ-μ and Kᵢ-σ₁>=1000 nM
++ One Kᵢ<1000 nM while the other Kᵢ is >=1000 nM
+++ Both Kᵢ-μ and Kᵢ-σ₁<1000 nM
++++ Both Kᵢ-μ and Kᵢ-σ₁<500 nM

All compounds prepared in the present application exhibit binding to the σ₁ receptor and the μ-opioid receptor, in particular the following binding results are shown:

| EX | σ1 and μ binding |
|---|---|
| 1 | + |
| 2 | ++ |
| 3 | ++ |
| 4 | ++ |
| 5 | ++ |
| 6 | +++ |
| 7 | + |
| 8 | ++ |
| 9 | + |

1-15. (canceled)

16. A compound of general Formula (I): 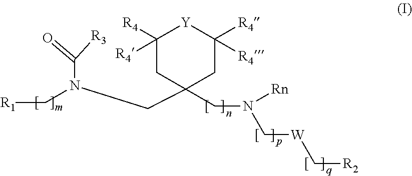
wherein
m is 0, 1, 2 or 3;
n is 0, 1, 2 or 3;
p is 1, 2 or 3;
q is 0, 1, 2 or 3;
Y is selected from the group consisting of —O— and —S—;
W is selected from the group consisting of —C(R_{w}R_{w′})—, —N(R_{w})— and —O—;
wherein R_{w} is selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl and substituted or unsubstituted alkylheterocyclyl;
R_{w′} is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₁ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
R₂ is selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
R₃ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl, substituted or unsubstituted alkylcycloalkyl, substituted or unsubstituted alkylaryl and substituted or unsubstituted alkylheterocyclyl;
R₄ and R_{4′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
alternatively, R₄ and R_{4′}, together with the carbon atom to which they are attached, form a substituted or unsubstituted cycloalkyl;
R_{4″} and R_{4′″} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
alternatively, R_{4″} and R_{4′″}, together with the carbon atom to which they are attached, form a substituted or unsubstituted cycloalkyl;
Rₙ is selected from the group consisting of hydrogen, substituted or unsubstituted C₁₋₆ alkyl, substituted or unsubstituted C₂₋₆ alkenyl and substituted or unsubstituted C₂₋₆ alkynyl;
optionally as a stereoisomer, including enantiomers and diastereomers, a racemate or as a mixture of two stereoisomers, including enantiomers and/or diastereomers, in any mixing ratio, or a corresponding salt thereof, or a corresponding solvate thereof.

17. The compound according to claim 16, wherein the compound of Formula (I) is a compound of Formula (I′) 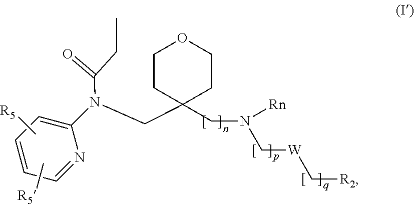
a compound of Formula (I²′) 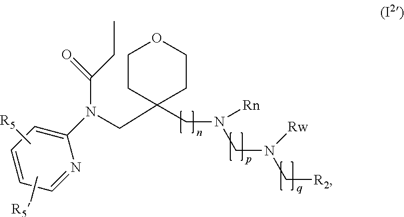
a compound of Formula (I³′) 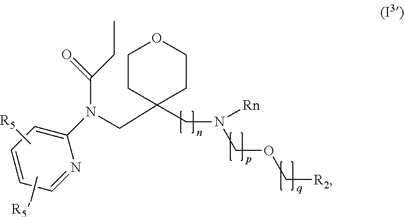
or a compound of Formula (I⁴′) 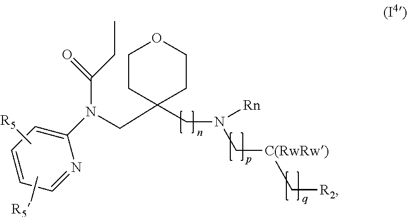
wherein R₅ and R_{5′} are independently selected from the group consisting of hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
R₁₁, R_{11′} and R_{11″} are independently selected from the group consisting of hydrogen, unsubstituted C₁₋₆ alkyl, unsubstituted C₂₋₆ alkenyl and unsubstituted C₂₋₆ alkynyl; and
R₂, Rₙ, R_{w}, R_{w′}, W, n, p, and q are as defined in claim 16.

18. The compound according to claim 16, wherein R₁ is 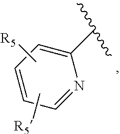
wherein R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
and R₁₁, R_{11′} and R_{11″} are independently selected from hydrogen, unsubstituted C₁₋₆ alkyl, unsubstituted C₂₋₆ alkenyl and unsubstituted C₂₋₆ alkynyl.

19. The compound according to claim 18, wherein R₁ is

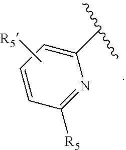

20. The compound according to claim 18, wherein R₁ is

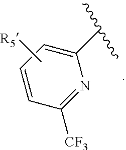

21. The compound according to claim 18, wherein R₁ is

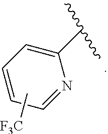

22. The compound according to claim 18, wherein R₁ is

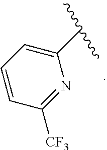 [SMILES: *c1cccc(C(F)(F)F)n1]

23. The compound according to claim 17, wherein 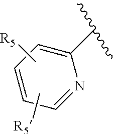
is selected from the group consisting of 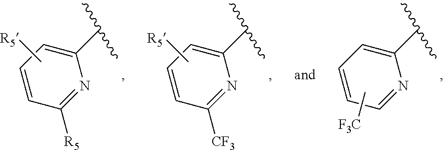
wherein R₅ and R_{5′} are independently selected from hydrogen, halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OR₁₁, —NR₁₁S(O)₂NR_{11′}R_{11″} and C(CH₃)₂OR₁₁;
and R₁₁, R_{11′} and R_{11″} are independently selected from hydrogen, unsubstituted C₁₋₆ alkyl, unsubstituted C₂₋₆ alkenyl and unsubstituted C₂₋₆ alkynyl.

24. The compound according to claim 23, wherein

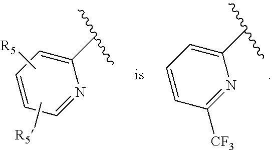

25. The compound according to claim 16, wherein R₂ is selected from the group consisting of hydrogen, substituted or unsubstituted methyl, substituted or unsubstituted ethyl, substituted or unsubstituted isopropyl and substituted or unsubstituted phenyl.

26. The compound according to claim 16, wherein R_{w} is selected from the group consisting of hydrogen, substituted or unsubstituted methyl and substituted or unsubstituted benzyl.

27. The compound according to claim 16, wherein n is 0, 2 or 3.

28. The compound according to claim 16, wherein p is 1 or 2.

29. The compound according to claim 16, wherein q is 0 or 1.

30. The compound according to claim 16, wherein the compound is selected from the group consisting of:
N-((4-((2-(benzyl(isobutyl)amino)ethyl)(methyl)amino)tetrahydro-2H-pyran-4-yl)methyl)-N-(6-(trifluoromethyl)pyridin-2-yl)propionamide,
N-((4-((2-(benzyl(methyl)amino)ethyl)(methyl)amino)tetrahydro-2H-pyran-4-yl)methyl)-N-(6-(trifluoromethyl)pyridin-2-yl)propionamide,
N-((4-((2-(isobutylamino)ethyl)(methyl)amino)tetrahydro-2H-pyran-4-yl)methyl)-N-(6-(trifluoromethyl)pyridin-2-yl)propionamide,
N-((4-((2-((2-ethoxyethyl)(methyl)amino)ethyl)(methyl)amino)tetrahydro-2H-pyran-4-yl)methyl)-N-(6-(trifluoromethyl)pyridin-2-yl)propionamide,
N-((4-(3-(isobutylamino)propyl)tetrahydro-2H-pyran-4-yl)methyl)-N-(6-(trifluoromethyl)pyridin-2-yl)propionamide,
N-((4-(3-((2-ethoxyethyl)amino)propyl)tetrahydro-2H-pyran-4-yl)methyl)-N-(6-(trifluoromethyl)pyridin-2-yl)propionamide,
N-((4-(2-(isobutylamino)ethyl)tetrahydro-2H-pyran-4-yl)methyl)-N-(6-(trifluoromethyl)pyridin-2-yl)propionamide,
N-((4-(3-(isobutyl(methyl)amino)propyl)tetrahydro-2H-pyran-4-yl)methyl)-N-(6-(trifluoromethyl)pyridin-2-yl)propionamide and
N-((4-(3-((2-ethoxyethyl)(methyl)amino)propyl)tetrahydro-2H-pyran-4-yl)methyl)-N-(6-(trifluoromethyl)pyridin-2-yl)propionamide.

31. A process for the preparation of the compound of Formula (I) according to claim 16,
a) wherein n is 0, said process comprises the acylation of the NH group of compounds VII 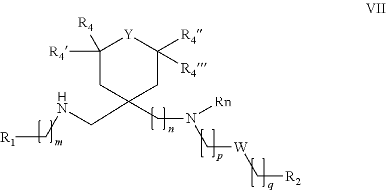
with an acyl halide of formula VIIIa or with an anhydride of formula VIIIb 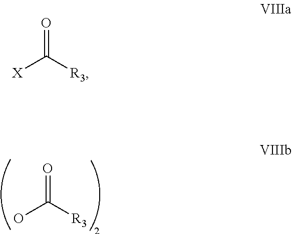
or
b) wherein n is 1, 2 or 3, said process comprises treating a compound of general formula XV 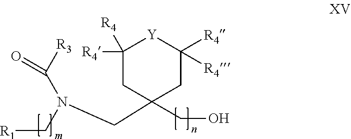
with trifluoromethanesulfonic anhydride, in the presence of a base, in a suitable solvent, including dichloromethane, at a suitable temperature, including at −78° C., and subsequent reaction of the triflate intermediate with an amino derivative of formula III 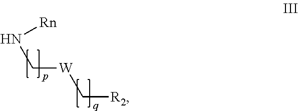
wherein L is a leaving group, including halogen and triflate, X is halogen, and, unless otherwise defined, R₁, R₂, R₃, R₄, R_{4′}, R_{4″}, R_{4′″}, Rₙ, R_{w}, R_{w′}, m, n, p, q, Y and W are as defined in claim 16.

32. A process for the preparation of the compound according of formula (I) according to claim 16, employing a compound of Formula II, III, IV, V, VI, VII, VIIIa, VIIIb, IX, X, XI, XII, XIII, XIV or XV: 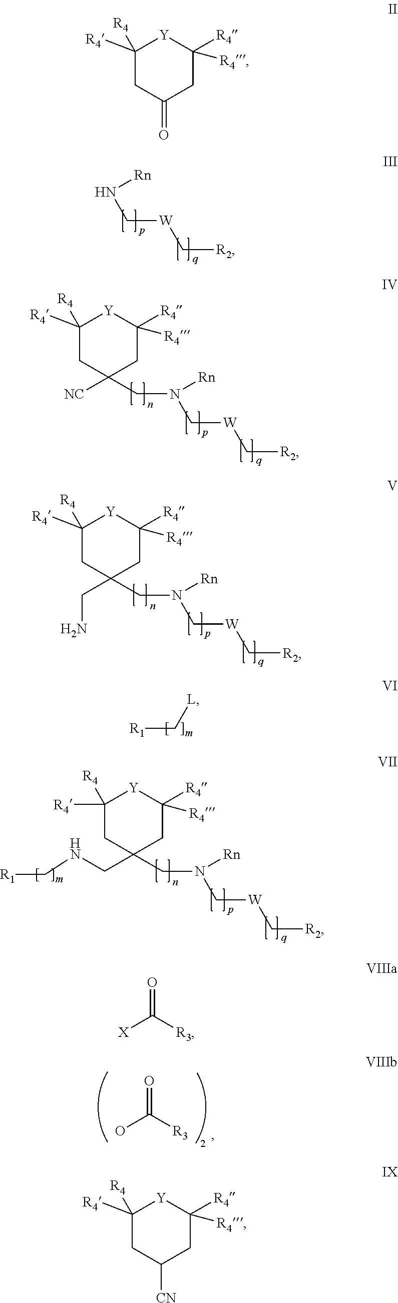 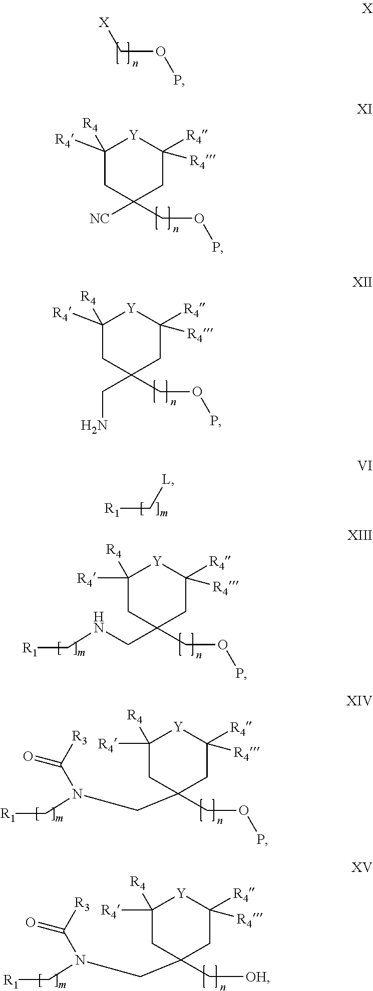
wherein L is a leaving group, including halogen and triflate, X is halogen, P is a protecting group, including benzyl, and R₁, R₂, R₃, R₄, R_{4′}, R_{4″}, R_{4″′}, Rₙ, R_{w}, R_{w′}, m, n, p, q, Y and W are as defined in claim 16.

33. A pharmaceutical composition which comprises the compound according to claim 16, or a pharmaceutically acceptable salt thereof, and a pharmaceutically acceptable carrier, adjuvant or vehicle.

34. A method of treating pain in a subject in need thereof, comprising administration of an effective amount of the compound according to claim 16.

35. The method according to claim 34, wherein the pain is selected from the group consisting of medium to severe pain, visceral pain, chronic pain, cancer pain, migraine, inflammatory pain, acute pain or neuropathic pain, allodynia and hyperalgesia.
